SEQUENCE LISTING (64 sequences)

SEQ ID NO: 1 - protein, 243 aa, synthetic construct
MGILPSPGMPALLSLVSLLSVLLMGCVAETGAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANNKEKEVLVLWGVHHPPNIGDQRALYHTENAYVSVVSSHYSRKFTPEIAKRPKVRDQEGRINYYWTLLEPGDTIIFEANGNLIAPRYAFALSRG

SEQ ID NO: 2 - protein, 243 aa, synthetic construct
MGILPSPGMPALLSLVSLLSVLLMGCVAETGAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANRGEKEVLVLWGVHHPPNIGDQRALYHTENAYVSVVSSHYSRKFTPEIAKRPKVRDQEGRINYYWTLLEPGDTIIFEANGNLIAPRYAFALSRG

SEQ ID NO: 3 - protein, 243 aa, synthetic construct
MGILPSPGMPALLSLVSLLSVLLMGCVAETGAPLQLGNCSVAGWILGNPECELLISRESWSYIVEKPNPENGTCFPGHFADYEELREQLSSVSSFERFNIFPKESSWPNHTTTGVSASCSHNGESSFYKNLLWLTGKNGLYPNLSKSYANRGEKEVLVLWGVHHPPNIGDQRALYHTENAYVSVVSSHYSRKFTPEIAKRPKVRDQEGRINYYWTLLEPGDTIIFEANGNLIAPRYAFALSRG

SEQ ID NO: 4 - protein, 243 aa, synthetic construct
MGILPSPGMPALLSLVSLLSVLLMGCVAETGAPLQLGNCSVAGWILGNPECELLIQDGSWSYIVEKPNPENGTCFPGHFADYEELREQLSSVSSFERFNIFPKESSWPNHTTTGVSASCSHNGESSFYKNLLWLTGKNGLYPNLSKSYANRGEKEVLVLWGVHHPPNIGDQRALYHTENAYVSVVSSHYSRKFTPEIAKRPKVRDQEGRINYYWTLLEPGDTIIFEANGNLIAPRYAFALSRG

SEQ ID NO: 5 - protein, 421 aa, synthetic construct
MGILPSPGMPALLSLVSLLSVLLMGCVAETGMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGSGGSGGSGGGAPLQLGNCSVAGWILGNPECELLISRESWSYIVEKPNPENGTCFPGHFADYEELREQLSSVSSFERFEIFPKESSWPNHTTTGVSASCSHNGESSFYKNLLWLTGKNGLYPNLSKSYANRGEKEVLVLWGVHHPPNIGDQRALYHTENAYVSVVSSHYSRKFTPEIAKRPKVRDQEGRINYYWTLLEPGDTIIFEANGNLIAPRYAFALSRG

SEQ ID NO: 6 - protein, 424 aa, synthetic construct
MGILPSPGMPALLSLVSLLSVLLMGCVAETGAPLQLGNCSVAGWILGNPECELLISRESWSYIVEKPNPENGTCFPGHFADYEELREQLSSVSSFERFEIFPKESSWPNHTTTGVSASCSHNGESSFYKNLLWLTGKNGLYPNLSKSYANRGEKEVLVLWGVHHPPNIGDQRALYHTENAYVSVVSSHYSRKFTPEIAKRPKVRDQEGRINYYWTLLEPGDTIIFEANGNLIAPRYAFALSRGGGSGGSGGSGGSGGSLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 7 - protein, 424 aa, synthetic construct
MGILPSPGMPALLSLVSLLSVLLMGCVAETGEPLQLGNCSVAGWILGNPECEHLISRESWSSIIEKPNPENGTCFPGHFADYENLTENLSSVSSFERFEIFPKESSWPNHTTTGVSASCSHNGESSFYKNLLWLTGKNGLYPNLSKSYANNKEKEVLVLWGVHHPPNIGDQRALYHTENAYVSVNSSHYNRTFTPEIAKRPKVRDQEGRINYYWTLLEPGDTIIFEANGNLIAPRYAFALSRGGGSGGSGGSGGSGGSLSKDIIKLLNEQVNKEMQSSNLYMSMSSWCYTHSLDGAGLFLFDHAAEEYEHAKKLIIFLNENNVPVNLTSISAPEHNFTGLTQIFQKAYEHEQNISESINNITDHAIKSKDHATFNFLQWYVAEQHEEEVLFKDILDKIELIGNENHGLYLADQYVKGIAKSRKS

SEQ ID NO: 8 - protein, 298 aa, synthetic construct
MGILPSPGMPALLSLVSLLSVLLMGCVAETGVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYV

SEQ ID NO: 9 - protein, 298 aa, synthetic construct
MGILPSPGMPALLSLVSLLSVLLMGCVAETGVQNSTTGKICNNPHRILDGKNCTLIDALLGDPHCDNFSNETWDLFVERSKAYSNCYPYDVPDYASLRSLVASSGTLEFINESFTWTGVTQNGGSNACKRGPGSGFFSRLNWLTKNGTTYPVLNVTMPNNDSFDKLYIWGVHHPSTNQEQTNLYVQASGNVTVSTRNSTQTIIPNIGSRPWVRGLSSRISIYWTIVKPGDNLTINSNGNLIAPRGYFKMRTGNSSIMRSDAPIDTCNSSCITPNGSIPNDKPEQNVSNISYGACPKYS

SEQ ID NO: 10 - protein, 298 aa, synthetic construct
MGILPSPGMPALLSLVSLLSVLLMGCVAETGVQSSSTGKICNNPHRILDGKNCTLIDALLGDPHCDNFSNETWDLFVERSKAYSNCYPYDVPDYASLRSLVASSGTLEFINESFTWTGVTQNGGSNACKRGPGSGFFSRLNWLTKNGTTYPVLNVTMPNNDSFDKLYIWGVHHPSTNQEQTNLYVQASGNVTVSTRNSTQTIIPNIGSRPWVRGLSSRISIYWTIVKPGDNLTINSNGNLIAPRGYFKMRTGNSSIMRSDAPIDTCISECITPNGSIPNDKPFQNVNKITYGACPKYS

SEQ ID NO: 11 - protein, 298 aa, synthetic construct
MGILPSPGMPALLSLVSLLSVLLMGCVAETGVQSSSTGKICNNPHRILDGIDCTLIDALLGDPHCDVFQNETWDLFVERSKAFSNCFPYDVPDYASLRSLVASSGTLKFITEGFYWTGVTQNGGSNACKRGPGSGFFSRLNWLTKSGSTYPVLNVSMPNHDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYV

SEQ ID NO: 12 - protein, 298 aa, synthetic construct
MGILPSPGMPALLSLVSLLSVLLMGCVAETGVQNSTSGSICLRPWRILDGKNCTLIDALLGDPHCDNFSNSTWDLFVERSKAYSDCFPYDVPRYEDLRSLVAKSGTLKFITEGFYWTGVTQNGTSNACKRGPNSSFFSRLNWLTKNGTSYPQLNVSMPNHDNFDKLYIWGVHHPSTNQEQTNLYVAESGNVTVSTSNSTQTIIPNIGSRPWVRGLSSRISIYWTIVKPGDVLVINSTGNLIAPRGYFKMREGNSSIMRSDAPTTNCNSSCITPNGSIDNSQPEQNVSNISYGDCPKPS

SEQ ID NO: 13 - protein, 476 aa, synthetic construct
MGILPSPGMPALLSLVSLLSVLLMGCVAETGMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGSGGSGGSGGGVQNSTTGKICNNPHRILDGKNCTLIDALLGDPHCDNFSNETWDLFVERSKAYSNCFPYDVPDYASLRSLVASSGTLEFINESFTWTGVTQNGGSNACKRGPGSGFFSRLNWLTKNGTTYPVLNVTMPNNDSFDKLYIWGVHHPSTNQEQTNLYVQASGNVTVSTRNSTQTIIPNIGSRPWVRGLSSRISIYWTIVKPGDNLTINSNGNLIAPRGYFKMRTGNSSIMRSDAPIDTCNSSCITPNGSIPNDKPEQNVSNISYGACPKYS

SEQ ID NO: 14 - protein, 479 aa, synthetic construct
MGILPSPGMPALLSLVSLLSVLLMGCVAETGVQNSTTGKICNNPHRILDGKNCTLIDALLGDPHCDNFSNETWDLFVERSKAYSNCYPYDVPDYASLRSLVASSGTLEFINESFTWTGVTQNGGSNACKRGPGSGFFSRLNWLTKNGTTYPVLNVTMPNNDSFDKLYIWGVHHPSTNQEQTNLYVQASGNVTVSTRNSTQTIIPNIGSRPWVRGLSSRISIYWTIVKPGDNLTINSNGNLIAPRGYFKMRTGNSSIMRSDAPIDTCNSSCITPNGSIPNDKPEQNVSNISYGACPKYSGGSGGSGGSGGSGGSLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 15 - protein, 464 aa, synthetic construct
MGILPSPGMPALLSLVSLLSVLLMGCVAETGVQNSTTGKICNNPHRILDGKNCTLIDALLGDPHCDNFSNETWDLFVERSKAYSNCYPYDVPDYASLRSLVASSGTLEFINESFTWTGVTQNGGSNACKRGPGSGFFSRLNWLTKNGTTYPVLNVTMPNNDSFDKLYIWGVHHPSTNQEQTNLYVQASGNVTVSTRNSTQTIIPNIGSRPWVRGLSSRISIYWTIVKPGDNLTINSNGNLIAPRGYFKMRTGNSSIMRSDAPIDTCNSSCITPNGSIPNDKPEQNVSNISYGACPKYSGGSGGSGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGF

SEQ ID NO: 16 - protein, 474 aa, synthetic construct
MGILPSPGMPALLSLVSLLSVLLMGCVAETGVQSSSTGKICNNPHRILDGKNCTLIDALLGDPHCDNFSNETWDLFVERSKAYSNCYPYDVPDYASLRSLVASSGTLEFINESFTWTGVTQNGGSNACKRGPGSGFFSRLNWLTKNGTTYPVLNVTMPNNDSFDKLYIWGVHHPSTNQEQTNLYVQASGNVTVSTRNSTQTIIPNIGSRPWVRGLSSRISIYWTIVKPGDNLTINSNGNLIAPRGYFKMRTGNSSIMRSDAPIDTCISECITPNGSIPNDKPFQNVNKITYGACPKYSGGSGGSGGSGGSGGSGGSGGSGGSVFEGHLVGTGLKVGVVVGRFNEFITSKLLGGALDGLKRHGVEENDIDVAWVPGAFEIPLIAKKMANSGKYDAVITLGTVIRGATTHYDYVANEVAKGVASLSLQTDIPVIFGVLTTETIEQAIERAGTKAGNKGYESAVAAIEMAHLSKHWA

SEQ ID NO: 17 - protein, 512 aa, synthetic construct
MGILPSPGMPALLSLVSLLSVLLMGCVAETGVQSSSTGKICNNPHRILDGKNCTLIDALLGDPHCDNFSNETWDLFVERSKAYSNCYPYDVPDYASLRSLVASSGTLEFINESFTWTGVTQNGGSNACKRGPGSGFFSRLNWLTKNGTTYPVLNVTMPNNDSFDKLYIWGVHHPSTNQEQTNLYVQASGNVTVSTRNSTQTIIPNIGSRPWVRGLSSRISIYWTIVKPGDNLTINSNGNLIAPRGYFKMRTGNSSIMRSDAPIDTCISECITPNGSIPNDKPFQNVNKITYGACPKYSGGSGGSGGSGGSGGSGGSGGSGGSASPIESARIGEVKRETKETNVSVKINLDGHGVSDSSTGIPFLDHMLDQLASHGLFDVHVRATGDTHIDDHHTNEDVALAIGTALLKALGERKGINRFGDFTAPLDEALIHVSLDLSGRPYLGYNLEIPTQRVGTYDTQLVEHFFQSLVDTSGMTLHIRQLAGKNSHHIIEATFKAFARALRQATESDPRR

SEQ ID NO: 18 - protein, 462 aa, synthetic construct
MGILPSPGMPALLSLVSLLSVLLMGCVAETGVQSSSTGKICNNPHRILDGKNCTLIDALLGDPHCDNFSNETWDLFVERSKAYSNCYPYDVPDYASLRSLVASSGTLEFINESFTWTGVTQNGGSNACKRGPGSGFFSRLNWLTKNGTTYPVLNVTMPNNDSFDKLYIWGVHHPSTNQEQTNLYVQASGNVTVSTRNSTQTIIPNIGSRPWVRGLSSRISIYWTIVKPGDNLTINSNGNLIAPRGYFKMRTGNSSIMRSDAPIDTCISECITPNGSIPNDKPFQNVNKITYGACPKYSGGSGGSGGSGGSGGSSNAMSHDVKELIEGLNEDLAGEYSAIIMYNHNAATVSGIYRQVLKPFFESEISDEQGHALYLAEKIKTLGGTPTTIPLRVKQAEDVREMLEYARQSEYETIKRYEKRKEQAANLNMTELVVKLEDMIADETNHMEELDRLLNDKAMVLN

SEQ ID NO: 19 - protein, 643 aa, synthetic construct
MGILPSPGMPALLSLVSLLSVLLMGCVAETGVQSSSTGKICNNPHRILDGKNCTLIDALLGDPHCDNFSNETWDLFVERSKAYSNCYPYDVPDYASLRSLVASSGTLEFINESFTWTGVTQNGGSNACKRGPGSGFFSRLNWLTKNGTTYPVLNVTMPNNDSFDKLYIWGVHHPSTNQEQTNLYVQASGNVTVSTRNSTQTIIPNIGSRPWVRGLSSRISIYWTIVKPGDNLTINSNGNLIAPRGYFKMRTGNSSIMRSDAPIDTCISECITPNGSIPNDKPFQNVNKITYGACPKYSGGSGGSGGSGGSGGSKELIRKLTEAFGPSGREEEVRSIILEELEGHIDGHRIDGLGNLIVWKGSGEKKVILDAHIDEIGVVVTNVDDKGFLTIEPVGGVSPYMLLGKRIRFENGTIGVVGMEGETTEERQENVRKLSFDKLFIDIGANSREEAQKMAPIGSFGVYDSGFVEVSGKYVSKAMDDRIGAAVIVEVFKRIKPAVTLYGVFSVQEEVGLVGASVAGYGVPADEAIAIDVTDSADTPKAIKRHAMRLSGGPALKVKDRASISSKRILENLIEIAEKFDIKYQMEVLTFGGTNAMGYQRTREGIPSATVSIPTRYVHSPSEMIAPDDVEATVDLLIRYLGA

SEQ ID NO: 20 - protein, 480 aa, synthetic construct
MGILPSPGMPALLSLVSLLSVLLMGCVAETGVQSSSTGKICNNPHRILDGKNCTLIDALLGDPHCDNFSNETWDLFVERSKAYSNCYPYDVPDYASLRSLVASSGTLEFINESFTWTGVTQNGGSNACKRGPGSGFFSRLNWLTKNGTTYPVLNVTMPNNDSFDKLYIWGVHHPSTNQEQTNLYVQASGNVTVSTRNSTQTIIPNIGSRPWVRGLSSRISIYWTIVKPGDNLTINSNGNLIAPRGYFKMRTGNSSIMRSDAPIDTCISECITPNGSIPNDKPFQNVNKITYGACPKYSGGSGGSGGSGGSGGSMKILVIQGPNLNMLGHRDPRLYGMVTLDQIHEIMQTFVKQGNLDVELEFFQTNFEGEIIDKIQESVGSDYEGIIINPGAFSHTSIAIADAIMLAGKPVIEVHLTNIQAREEFRKNSYTGAAAGGVIMGFGPLGYNMALMAMVNILAEMKAFQEAQKNNPNNPINNQK

SEQ ID NO: 21 - protein, 458 aa, synthetic construct
MGILPSPGMPALLSLVSLLSVLLMGCVAETGVQSSSTGKICNNPHRILDGKNCTLIDALLGDPHCDNFSNETWDLFVERSKAYSNCYPYDVPDYASLRSLVASSGTLEFINESFTWTGVTQNGGSNACKRGPGSGFFSRLNWLTKNGTTYPVLNVTMPNNDSFDKLYIWGVHHPSTNQEQTNLYVQASGNVTVSTRNSTQTIIPNIGSRPWVRGLSSRISIYWTIVKPGDNLTINSNGNLIAPRGYFKMRTGNSSIMRSDAPIDTCISECITPNGSIPNDKPFQNVNKITYGACPKYSGGSGGSGGSGGSGGSAQIHVLVLHGPNLNLLGRREPDHYGRTTLAEIDARLKTEAEGRGWLLHSLQSNAEHILVDAVQQAPTQGVTHIVLNPAAFTHTSVALRDALAAVAIPFIEVHLSNIHAREPFRRHSYFSDIASGLITGLGAEGYSLALDAIARRF

SEQ ID NO: 22 - protein, 460 aa, synthetic construct
MGILPSPGMPALLSLVSLLSVLLMGCVAETGVQSSSTGKICNNPHRILDGKNCTLIDALLGDPHCDNFSNETWDLFVERSKAYSNCYPYDVPDYASLRSLVASSGTLEFINESFTWTGVTQNGGSNACKRGPGSGFFSRLNWLTKNGTTYPVLNVTMPNNDSFDKLYIWGVHHPSTNQEQTNLYVQASGNVTVSTRNSTQTIIPNIGSRPWVRGLSSRISIYWTIVKPGDNLTINSNGNLIAPRGYFKMRTGNSSIMRSDAPIDTCISECITPNGSIPNDKPFQNVNKITYGACPKYSGGSGGSGGSGGSGGSMSELIVNVINGPNLGRLGRREPAVYGGTTHDELVALIEREAAELGLKAVVRQSDSEAQLLDWIHQAADAAEPVILNAGGLTHTSVALRDAAAELSAPLIEVHISNVHAREEFRRHSYLSPIATGVIVGLGIQGYLLALRYLAEHVGT

SEQ ID NO: 23 - protein, 31 aa, synthetic construct
MGILPSPGMPALLSLVSLLSVLLMGCVAETG

SEQ ID NO: 24 - protein, 9 aa, synthetic construct
GTKHHHHHH

SEQ ID NO: 25 - protein, 30 aa, synthetic construct
GTKHHHHHHGGSGGSGLNDIFEAQKIEWHE

SEQ ID NO: 26 - protein, 5 aa, synthetic construct
GGGGS

SEQ ID NO: 27 - protein, 10 aa, synthetic construct
GGGGSGGGGS

SEQ ID NO: 28 - protein, 15 aa, synthetic construct
GGGGSGGGGSGGGGS

SEQ ID NO: 29 - protein, 20 aa, synthetic construct
GGGGSGGGGSGGGGSGGGGS

SEQ ID NO: 30 - protein, 25 aa, synthetic construct
GGGGSGGGGSGGGGSGGGGSGGGGS

SEQ ID NO: 31 - protein, 30 aa, synthetic construct
GGGGSGGGGSGGGGSGGGGSGGGGSGGGGS

SEQ ID NO: 32 - protein, 35 aa, synthetic construct
GGGGSGGGGSGGGGSGGGGSGGGGSGGGGSGGGGS

SEQ ID NO: 33 - protein, 40 aa, synthetic construct
GGGGSGGGGSGGGGSGGGGSGGGGSGGGGSGGGGSGGGGS

SEQ ID NO: 34 - protein, 45 aa, synthetic construct
GGGGSGGGGSGGGGSGGGGSGGGGSGGGGSGGGGSGGGGSGGGGS

SEQ ID NO: 35 - protein, 50 aa, synthetic construct
GGGGSGGGGSGGGGSGGGGSGGGGSGGGGSGGGGSGGGGSGGGGSGGGGS

SEQ ID NO: 36 - protein, 55 aa, synthetic construct
GGGGSGGGGSGGGGSGGGGSGGGGSGGGGSGGGGSGGGGSGGGGSGGGGSGGGGS

SEQ ID NO: 37 - protein, 60 aa, synthetic construct
GGGGSGGGGSGGGGSGGGGSGGGGSGGGGSGGGGSGGGGSGGGGSGGGGSGGGGSGGGGS

SEQ ID NO: 38 - protein, 4 aa, synthetic construct
GGGS

SEQ ID NO: 39 - protein, 7 aa, synthetic construct
AEAAAKA

SEQ ID NO: 40 - protein, 15 aa, synthetic construct
EAAAKEAAAKEAAAK

SEQ ID NO: 41 - protein, 5 aa, synthetic construct
  REGION >5: note = The entire sequence of amino acids 1-5 can be repeated one to three times.
EAAAK

SEQ ID NO: 42 - protein, 42 aa, synthetic construct
AEAAKEAAKEAAKEAAKALEAEAAAKEAAAKEAAAKEAAAKA

SEQ ID NO: 43 - protein, 5 aa, synthetic construct
PAPAP

SEQ ID NO: 44 - protein, 12 aa, synthetic construct
AEAAAKEAAAKA

SEQ ID NO: 45 - protein, 20 aa, synthetic construct
  VARIANT 1..2: note = The amino acid residues 1-2 can be repeated one to thirty four times.
APAPAPAPAPAPAPAPAPAP

SEQ ID NO: 46 - protein, 17 aa, synthetic construct
VSQTSKLTRAETVFPDV

SEQ ID NO: 47 - protein, 6 aa, synthetic construct
PLGLWA

SEQ ID NO: 48 - protein, 6 aa, synthetic construct
RVLAEA

SEQ ID NO: 49 - protein, 10 aa, synthetic construct
EDVVCCSMSY

SEQ ID NO: 50 - protein, 7 aa, synthetic construct
GGIERGS

SEQ ID NO: 51 - protein, 10 aa, synthetic construct
TRHRQPRGWE

SEQ ID NO: 52 - protein, 10 aa, synthetic construct
AGNRVRRSVG

SEQ ID NO: 53 - protein, 9 aa, synthetic construct
RRRRRRRRR

SEQ ID NO: 54 - protein, 149 aa, synthetic construct
MVLILNGPNLNLLGRREPEVYGRTTLEELEALCEAWGAELGLGVVFRQTNYEGQLIEWVQNAWQEGFLAIVLNPGALTHYSYALLDAIRAQPLPVVEVHLTNLHAREEFRRHSVTAPAARGIVSGFGPLSYKLALVYLAETLEVGGEGF

SEQ ID NO: 55 - protein, 151 aa, synthetic construct
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGF

SEQ ID NO: 56 - protein, 6 aa, synthetic construct
GGSGGS

SEQ ID NO: 57 - protein, 9 aa, synthetic construct
GGSGGSGGS

SEQ ID NO: 58 - protein, 8 aa, synthetic construct
GGGSGGGS

SEQ ID NO: 59 - protein, 12 aa, synthetic construct
GGGSGGGSGGGS

SEQ ID NO: 60 - protein, 27 aa, unidentified
  source 1..27: note = Human immunodeficiency virus
KIFIMIVGGLIGLRIVFAVLSVIHRVR

SEQ ID NO: 61 - protein, 24 aa, Severe acute respiratory syndrome-related coronavirus
KWPWYIWLGFIAGLIAIVMVTIML

SEQ ID NO: 62 - protein, 22 aa, unidentified
  source 1..22: note = Vesicular stomatitis virus
KSSIASFFFIIGLIIGLFLVLR

SEQ ID NO: 63 - protein, 6 aa, synthetic construct
  VARIANT 1..3: note = The amino acid residues 1-3 can be repeated one or more times.
GGSGGS

SEQ ID NO: 64 - protein, 4 aa, synthetic construct
  REGION >4: note = The entire sequence of amino acids 1-4 can be repeated one or more times.
GGGS

(19) United States
(12) Patent Application Publication
(10) Pub. No.: US 2026/0295029 A1
(43) Pub. Date: Oct. 1, 2026

(54) INFLUENZA EHEAD IMMUNOGENS

(71) Applicant: The Scripps Research Institute, La Jolla, CA (US)

(72) Inventors: William SCHIEF, La Jolla, CA (US); Xiaozhen HU, La Jolla, CA (US)

(21) Appl. No.: 19/489,371

(22) PCT Filed: May 31, 2024
(86) PCT No.: PCT/US2024/031940

§ 371 Date: Dec. 2, 2025

Related U.S. Application Data
(60) Provisional application No. 63/470,625, filed on Jun. 2, 2023.

(51) Int. Cl. A61K 39/145; A61K 9/51; A61K 35/17; A61K 39/00; A61K 40/13; C07K 14/005; C12N 9/10; C12N 15/88

(52) U.S. Cl. CPC A61K 39/145; A61K 9/5169; A61K 35/17; A61K 40/13; C07K 14/005; C12N 9/1085; C12N 15/88; A61K 2039/53; A61K 2039/55555; C07K 2319/02; C07K 2319/91; C12N 2760/16122; C12N 2760/16134; C12Y 205/01078

(57) ABSTRACT

The present invention relates to influenza immunogens and a vaccine platform for influenza viruses. In particular, the invention relates to a non-naturally occurring polypeptides comprising an engineered hemagglutinin lateral patch sequence and a secretion signal sequence, nucleic acids encoding the same, vectors containing the nucleic acids, nanoparticles containing the polypeptides, nucleic acids or vectors, cells containing the nanoparticles, polypeptides, nucleic acids or vectors, pharmaceutical compositions comprising the cells, nanoparticles, polypeptides, nucleic acids or vectors, vaccines comprising the pharmaceutical compositions and methods for immunization with the vaccines.

### INCORPORATION BY REFERENCE

This application claims priority to U.S. provisional application Ser. No. 63/470,625, filed Jun. 2, 2023, which is incorporated by reference herein in its entirety.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy, was created Jul. 17, 2024, is named Y9054_99004SL.xml and is 67,754 bytes in size.

### FIELD OF THE INVENTION

The present invention relates to influenza immunogens and a vaccine platform for influenza viruses.

### BACKGROUND OF THE INVENTION

Current seasonal influenza vaccines mainly target the head domain of the hemagglutinin (HA) protein. However, this region is highly variable and undergoes frequent changes, allowing the virus to evade antibody recognition. As a result, current vaccines have limited efficacy against newly emerging viral strains.

The lateral patch (LP) of the head domain is highly conserved in H1N1 of the virus. Several lateral patch bnAbs such as CL6649, 4B06 and 2A04 (D. D. Raymond et al., Conserved epitope on influenza-virus hemagglutinin head defined by a vaccine-induced antibody. Proc Natl Acad Sci USA 115, 168-173 (2018) and J. J. Guthmiller et al., First exposure to the pandemic H1N1 virus induced broadly neutralizing antibodies targeting hemagglutinin head epitopes. Sci Transl Med 13, (2021)) have been identified and they show great breadth among H1N1 viruses. A similar lateral patch bnAb, C585, neutralizes multiple H3N2 strains isolated since 1968 (Y. Qiu et al., Mapping of a Novel H3-Specific Broadly Neutralizing Monoclonal Antibody Targeting the Hemagglutinin Globular Head Isolated from an Elite Influenza Virus-Immunized Donor Exhibiting Serological Breadth. J Virol 94, (2020)).

Citation or identification of any document in this application is not an admission that such document is available as prior art to the present invention.

### SUMMARY OF THE INVENTION

The present invention relates to influenza eHead immunogens targeting lateral patch broadly neutralizing antibodies (bnAbs).

Current vaccines have limited efficacy against newly emerging viral strains. Applicants developed vaccines with broad coverage for pan-subtype influenza virus by redirecting the immune response toward conserved epitope. In particular, Applicants developed novel vaccine candidates to assist the induction of broadly neutralizing antibodies against influenza group 1 (H1N1) and group 2 (H3N2). Targeting the lateral patch (LP) of the head domain leads to the development of more broadly protective vaccines that are effective against a wider range of influenza strains.

Applicants used structure-guided protein design to extract the head region from the hemagglutinin protein (HA) and developed a stabilized eHead H1 immunogen. The eHead H1 construct includes HA residues from 55-263. Applicants employed glycan-masking to develop eHead variants focused on lateral patch epitope. In order to prime lateral patch responses consistently, Applicants also applied structure-guided mammalian-display directed evolution to redesign the eHead H1 to target human naive precursors of lateral patch bnAbs, in which the human precursors were identified in next-generation sequencing (NGS) data of human naive antibody heavy chains. To generate potent and long-lasting immune responses, Applicants also developed single-component self-assembling nanoparticles to provide multivalent display of the eHead. Applicants developed multiple types of nanoparticles: lumazine synthase-based 60mers, ferritin-based 24mers, non-ferritin based 24mers and 12mers. These nanoparticles are amenable to delivery by mRNA or by purified protein.

Applicants used structure-guided protein design to extract the head region from the hemagglutinin protein (HA) and developed a stabilized eHead H3 immunogen. The eHead H3 construct includes HA residues from 43-309. Applicants employed computational design approach and glycan-masking to develop a stabilized eHead H3 that is well-folded and highly expressed. In order to prime C585-like H3 lateral patch responses consistently, Applicants applied structure-guided mammalian-display directed evolution to redesign the eHead H3 to target C585-like human naive precursors. Compared to eHead H1, eHead H3 is more difficult to incorporate into nanoparticles. After a few rounds of iterative optimization, Applicants produced different 12mer, 24mer, and 60mer NPs for eHead.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1 shows the SPR data of eH1_GT1.1 and eH1_GT1.22 against 18 lateral patch bnAb NGS precursors (Left panel) and 32 competitor NGS precursors (Right panel). GT1.1 binds 72% of the bnAb precursors with geomean dissociation constant (K_{D}) of 600 nM, and GT1.22 binds to 78% of the bnAb precursors with a geomean K_{D} of 200 nM. Neither shows detectable binding to any of the competitors.

FIG. 2 shows the SPR data of eH1_GT2.1 and GT1.22 binding to lateral patch bnAb NGS precursors. GT1.22 binds to 78% of the bnAb precursors with a geomean K_{D} of 200 nM. For the same set of NGS antibodies, eH1_GT2.1 binds to all of them with geomean K_{D} of 10 nM. For a larger random set of NGS bnAb precursors, GT2.1 binds 46% of them with geomean K_{D} of 77.8 nM.

FIG. 3 shows the size-exclusion traces after lectin purification, negative-stain electron microscopy images, and yields of 60mer nanoparticles. eHead_H1_GT1_NP_v1 (Left panel) is 60mer and eHead_H1_GT1_NP_v3 (right panel) is ferritin 24mer.

FIG. 4 SPR data of eH3_GT1.19 against 10 lateral patch bnAb (C585) precursors and bnAbs. GT1.19 shows no detectable binding to NGS precursors, but it improves binding to C585 CDR3mat_iGL by ~75 fold compared to eHead-H3HK68. GT1.19 also improves binding to the mature C585 by ~200 fold.

### DETAILED DESCRIPTION OF THE INVENTION

Where used herein and unless specifically indicated otherwise, the following terms are intended to have the following meanings in addition to any broader (or narrower) meanings the terms might enjoy in the art:

The term “isolated” or “non-naturally occurring” is used herein to indicate that the isolated moiety (e.g. peptide or compound) exists in a physical milieu distinct from that in which it occurs in nature. For example, the isolated peptide may be substantially isolated with respect to the complex cellular milieu in which it naturally occurs. The absolute level of purity is not critical, and those skilled in the art may readily determine appropriate levels of purity according to the use to which the peptide is to be put. The term “isolating” when used a step in a process is to be interpreted accordingly.

In many circumstances, the isolated moiety will form part of a composition (for example a more or less crude extract containing many other molecules and substances), buffer system, matrix or excipient, which may for example contain other components (including proteins, such as albumin).

In other circumstances, the isolated moiety may be purified to essential homogeneity, for example as determined by PAGE or column chromatography (for example HPLC or mass spectrometry). In preferred embodiments, the isolated peptide or nucleic acid of the invention is essentially the sole peptide or nucleic acid in a given composition.

In an advantageous embodiment, a tag may be utilized for purification or biotinylation. The tag for purification may be a his tag. In another embodiment, the tag for biotinylation may be an avi-tag. Other tags are contemplated for purification, however, purification may be accomplished without a tag. In another embodiment, antibody (such as, not limited to, a broadly neutralizing antibody) affinity columns are contemplated. In another embodiment, lectin columns are contemplated.

The proteins and compounds of the invention need not be isolated in the sense defined above, however.

The term “pharmaceutical composition” is used herein to define a solid or liquid composition in a form, concentration and level of purity suitable for administration to a patient (e.g. a human patient) upon which administration it may elicit the desired physiological changes. The terms “immunogenic composition” and “immunological composition” and “immunogenic or immunological composition” cover any composition that elicits an immune response against a targeted pathogen. Terms such as “vaccinal composition” and “vaccine” and “vaccine composition” cover any composition that induces a protective immune response against the targeted pathogen or which efficaciously protects against the pathogen or virus; for instance, after administration or injection, elicits a protective immune response against the targeted pathogen or virus or provides efficacious protection against the pathogen or virus. Accordingly, an immunogenic or immunological composition induces an immune response, which may, but need not, be a protective immune response. An immunogenic or immunological composition may be used in the treatment of individuals infected with the pathogen or virus, e.g., to stimulate an immune response against the pathogen, such as by stimulating antibodies against the pathogen or virus. Thus, an immunogenic or immunological composition may be a pharmaceutical composition. Furthermore, when the text speaks of “immunogen, antigen or epitope”, an immunogen may be an antigen or an epitope of an antigen. A diagnostic composition is a composition containing a compound or antibody, e.g., a labeled compound or antibody, that is used for detecting the presence in a sample, such as a biological sample, e.g., blood, semen, vaginal fluid, etc., of an antibody that binds to the compound or an immunogen, antigen or epitope that binds to the antibody; for instance, an antibody, immunogen, antigen or epitope.

A “conservative amino acid change” is one in which the amino acid residue is replaced with an amino acid residue having a similar side chain. Families of amino acid residues having similar side chains have been defined in the art. These families include amino acids with basic side chains (e.g. lysine, arginine and histidine), acidic side chains (e.g. aspartic acid and glutamic acid), non-charged amino acids or polar side chains (e.g. glycine, asparagine, glutamine, serine, threonine, tyrosine and cysteine), non-polar side chains (e.g. alanine, valine, leucine, isoleucine, proline, phenylalanine, methionine and tryptophan), beta-branched side chains (e.g. threonine, valine and isoleucine), and aromatic side chains (e.g. tyrosine, phenylalanine, tryptophan and histidine).

The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling or bioactive component.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The term “antibody” includes intact molecules as well as fragments thereof, such as Fab, F(ab′)₂, Fv and scFv which are capable of binding the epitope determinant. These antibody fragments retain some ability to selectively bind with its antigen or receptor and include, for example:
(a) Fab, the fragment which contains a monovalent antigen-binding fragment of an antibody molecule may be produced by digestion of whole antibody with the enzyme papain to yield an intact light chain and a portion of one heavy chain;
(b) Fab′, the fragment of an antibody molecule may be obtained by treating whole antibody with pepsin, followed by reduction, to yield an intact light chain and a portion of the heavy chain; two Fab′ fragments are obtained per antibody molecule;
(c) F(ab′)₂, the fragment of the antibody that may be obtained by treating whole antibody with the enzyme pepsin without subsequent reduction; F(ab′)₂ is a dimer of two Fab′ fragments held together by two disulfide bonds;
(d) scFv, including a genetically engineered fragment containing the variable region of a heavy and a light chain as a fused single chain molecule.

General methods of making these fragments are known in the art. (See for example, Harlow and Lane, Antibodies: A Laboratory Manual, Cold Spring Harbor Laboratory, New York (1988), which is incorporated herein by reference). Fabs, Fv and scFV may also be made recombinantly, i.e. expressed as Fab, Fv or scFV rather than cleaving an intact IgG.

A “neutralizing antibody” may inhibit the entry of a virus with a neutralization index >1.5 or >2.0. Broad and potent neutralizing antibodies may neutralize greater than about 50% of viruses (from diverse clades and different strains within a clade) in a neutralization assay. The inhibitory concentration of the monoclonal antibody may be less than about 25 mg/ml to neutralize about 50% of the input virus in the neutralization assay.

An “isolated antibody” or “non-naturally occurring antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “monoclonal antibody” as used herein refers to an antibody obtained from a population of substantially homogeneous antibodies, i.e., the individual antibodies which may comprise the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

An “antibody fragment” may comprise a portion of an intact antibody, preferably the antigen binding or variable region of the intact antibody. Examples of antibody fragments include Fab, Fab′, F(ab′)₂, scFV and Fv fragments; diabodies; linear antibodies (see U.S. Pat. No. 5,641,870; Zapata et al. 1995 Protein Eng. 8(10): 1057-1062); single-chain antibody molecules; and multispecific antibodies formed from antibody fragments.

It should be understood that the proteins, including the antibodies of the invention may differ from the exact sequences illustrated and described herein. Thus, the invention contemplates deletions, additions and substitutions to the sequences shown, so long as the sequences function in accordance with the methods of the invention. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated or non-naturally occurring replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the sequences illustrated and described but possessing minor amino acid substitutions that do not substantially affect the immunogenicity of the protein are, therefore, within the scope of the invention.

As used herein the terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid may be single-stranded, or partially or completely double-stranded (duplex). Duplex nucleic acids may be homoduplex or heteroduplex.

As used herein the term “transgene” may used to refer to “recombinant” nucleotide sequences that may be derived from any of the nucleotide sequences encoding the proteins of the present invention. The term “recombinant” means a nucleotide sequence that has been manipulated “by man” and which does not occur in nature, or is linked to another nucleotide sequence or found in a different arrangement in nature. It is understood that manipulated “by man” means manipulated by some artificial means, including by use of machines, codon optimization, restriction enzymes, etc.

For example, in one embodiment the nucleotide sequences may be mutated such that the activity of the encoded proteins in vivo is abrogated. In another embodiment the nucleotide sequences may be codon optimized, for example the codons may be optimized for human use. In preferred embodiments the nucleotide sequences of the invention are both mutated to abrogate the normal in vivo function of the encoded proteins, and codon optimized for human use. For example, each of the sequences of the invention, such as the mutant trimers, may be altered in these ways.

As regards codon optimization, the nucleic acid molecules of the invention have a nucleotide sequence that encodes the antigens of the invention and may be designed to employ codons that are used in the genes of the subject in which the antigen is to be produced. Many viruses use a large number of rare codons and, by altering these codons to correspond to codons commonly used in the desired subject, enhanced expression of the antigens may be achieved. In a preferred embodiment, the codons used are “humanized” codons, i.e., the codons are those that appear frequently in highly expressed human genes (Andre et al., J. Virol. 72:1497-1503, 1998) instead of those codons that are frequently used by a virus. Such codon usage provides for efficient expression of transgenic proteins in human cells. Any suitable method of codon optimization may be used. Such methods, and the selection of such methods, are well known to those of skill in the art. In addition, there are several companies that will optimize codons of sequences, such as Geneart (geneart.com). Thus, the nucleotide sequences of the invention may readily be codon optimized.

The invention further encompasses nucleotide sequences encoding functionally and/or antigenically equivalent variants and derivatives of the antigens of the invention and functionally equivalent fragments thereof. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the antigen, epitope, immunogen, peptide or polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 may be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms may be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

The various recombinant nucleotide sequences and antibodies of the invention are made using standard recombinant DNA and cloning techniques. Such techniques are well known to those of skill in the art. See for example, “Molecular Cloning: A Laboratory Manual”, second edition (Sambrook et al. 1989).

The nucleotide sequences of the present invention may be inserted into “vectors.” The term “vector” is widely used and understood by those of skill in the art, and as used herein the term “vector” is used consistent with its meaning to those of skill in the art. For example, the term “vector” is commonly used by those skilled in the art to refer to a vehicle that allows or facilitates the transfer of nucleic acid molecules from one environment to another or that allows or facilitates the manipulation of a nucleic acid molecule.

Any vector that allows expression of the proteins of the present invention may be used in accordance with the present invention. In certain embodiments, the proteins of the present invention may be used in vitro (such as using cell-free expression systems) and/or in cultured cells grown in vitro in order to produce encoded proteins, which may then be used for various applications such as in the production of proteinaceous vaccines. For such applications, any vector that allows expression of the proteins in vitro and/or in cultured cells may be used.

For applications where it is desired that the proteins be expressed in vivo, for example when the transgenes of the invention are used in DNA or DNA-containing vaccines, any vector that allows for the expression of the proteins of the present invention and is safe for use in vivo may be used. In preferred embodiments the vectors used are safe for use in humans, mammals and/or laboratory animals.

For the proteins of the present invention to be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” may be any nucleic acid element, such as, but not limited to promoters, enhancers, IRES, introns, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences of the invention lead to the expression of the encoded protein. The expression of the transgenes of the present invention may be under the control of a constitutive promoter or of an inducible promoter, which initiates transcription only when exposed to some particular external stimulus, such as, without limitation, antibiotics such as tetracycline, hormones such as ecdysone, or heavy metals. The promoter may also be specific to a particular cell-type, tissue or organ. Many suitable promoters and enhancers are known in the art, and any such suitable promoter or enhancer may be used for expression of the transgenes of the invention. For example, suitable promoters and/or enhancers may be selected from the Eukaryotic Promoter Database (EPDB).

The vectors used in accordance with the present invention should typically be chosen such that they contain a suitable gene regulatory region, such as a promoter or enhancer, such that the antibodies of the invention may be expressed.

Any suitable vector may be used depending on the application. For example, plasmids, viral vectors, bacterial vectors, protozoal vectors, insect vectors, baculovirus expression vectors, yeast vectors, mammalian cell vectors, and the like, may be used. Eucaryotic expression vectors are advantageous. Suitable vectors may be selected by the skilled artisan taking into consideration the characteristics of the vector and the requirements for expressing the proteins under the identified circumstances.

As used herein, broadly neutralizing antibodies are antibodies that neutralize more than one type, subtype and/or strain of influenza virus. For example, broadly neutralizing antibodies elicited against an HA protein from a Type A influenza virus may neutralize a Type B or Type C virus. As a further example, broadly neutralizing antibodies elicited against an HA protein from Group I influenza virus may neutralize a Group 2 virus. As an additional example, broadly neutralizing antibodies elicited against an HA protein from one sub-type or strain of virus, may neutralize another sub-type or strain of virus. For example, broadly neutralizing antibodies elicited against an HA protein from an H1 influenza virus may neutralize viruses from one or more sub-types selected from the group consisting of H2, H3, H4, H5, H6, H7, H8, H8, H10, H11, H12, H13, H14, H15, H16, H17 or H18.

According to the present invention all nomenclature used to classify influenza virus is that commonly used by those skilled in the art. Thus, a Type, or Group, of influenza virus refers to influenza Type A, influenza Type B or influenza type C. It is understood by those skilled in the art that the designation of a virus as a specific Type relates to sequence difference in the respective M1 (matrix) protein or NP (nucleoprotein). Type A influenza viruses are further divided into Group1 and Group 2. These Groups are further divided into subtypes, which refers to classification of a virus based on the sequence of its HA protein. Examples of current commonly recognized subtypes are H1, H2, H3, H4, H5, H6, H7, H8, H8, H10, H11, H12, H13, H14, H15, H16, H17 or H18. Group 1 influenza subtypes are H1, H2, H5, H6, H8, H9, H11, H12, H13, H16, H17 and H18. Group 2 influenza subtypes are H3, H4, H7, H10, H14, and H15. Finally, the term strain refers to viruses within a subtype that differ from one another in that they have small, genetic variations in their genome.

As used herein, an influenza hemagglutinin protein, or HA protein, refers to a full-length influenza hemagglutinin protein or any portion thereof, that is useful for producing protein constructs and nanoparticles of the invention or that are capable of eliciting an immune response. Preferred HA proteins are those that are capable of forming a trimer. An epitope of a full-length influenza HA protein refers to a portion of such protein that can elicit an antibody response against the homologous influenza strain, i.e., a strain from which the HA is derived. In some embodiments, such an epitope can also elicit an antibody response against a heterologous influenza strain, i.e., a strain having an HA that is not identical to that of the HA of the immunogen. In some embodiments, the epitope elicits a broadly heterosubtypic protective response. In some embodiments, the epitope elicits neutralizing antibodies.

In one embodiment, the HA protein comprises at least one immunogenic portion from the stem region of influenza HA protein, wherein the protein elicits protective antibodies against an influenza virus. In one embodiment, the HA protein comprises at least one immunogenic portion from the stem region of an HA protein from a virus selected from the group consisting of influenza type A viruses, influenza type B viruses and influenza type C viruses, wherein the protein elicits protective antibodies against an influenza virus. In one embodiment, the HA protein comprises at least one immunogenic portion from the stem region of an HA protein selected from the group consisting of an H1 influenza virus HA protein, an H2 influenza virus HA protein, an influenza H3 virus HA protein, an influenza H4 virus HA protein, an influenza H5 virus HA protein, an influenza H6 virus HA protein, an H7 influenza virus HA protein, an H8 influenza virus HA protein, an H9 influenza virus HA protein, an H10 influenza virus HA protein HA protein, an H11 influenza virus HA protein, an H12 influenza virus HA protein, an H13 influenza virus HA protein, an H14 influenza virus HA protein, an H15 influenza virus HA protein, an H16 influenza virus HA protein, an H17 influenza virus HA protein, and an H18 influenza virus HA protein.

The HA protein may be from a virus selected from the group consisting of influenza A/New Caledonia/20/1999 (1999 NC, H1), A/California/04/2009 (2009 CA, H1), A/Singapore/1/1957 (1957 Sing, H2), A/Hong Kong/1/1968 (1968 HK, H3), A/Brisbane/10/2007 (2007 Bris, H3), A/Indonesia/05/2005 (2005 Indo, H5), B/Florida/4/2006 (2006 Flo, B), A/Perth/16/2009 (2009 Per, H3), A/Brisbane/59/2007 (2007 Bris, H1), B/Brisbane/60/2008 (2008 Bris, B).

In some embodiments, the present invention comprises a non-naturally occurring polypeptide comprising an engineered hemagglutinin lateral patch sequence and a secretion signal sequence.

In some embodiments, the secretion signal sequence and the hemagglutinin lateral patch sequence are operably linked. In some embodiments, the secretion signal sequence is N-terminal to the lateral patch sequence. In some embodiments, the secretion signal sequence is C-terminal to the lateral patch sequence. In some embodiments, the secretion signal sequence is any secretion signal sequence known in the art. In some embodiments, the secretion signal sequence is a viral signal sequence.

The term “secretion signal sequence” refers to an amino acid sequence or a nucleotide sequence coding for a peptide at the N-terminus or C-terminus of the translation product that is responsible for directing secretory proteins into the secretion pathway. Examples of suitable secretion signal sequences for use in the present invention comprise but are not limited to mammalian signal sequences, Drosophila signal sequences, bacterial signal sequences, and viral signal sequences as described in signalpeptide.de.

In some embodiments, the secretion signal sequence comprises 70%, 75%, 8%, 85%, 90%, 95%, or 99% identity to MGILPSPGMPALLSLVSLLSVLLMGCVAETG. In some embodiments the secretion signal sequence is MGILPSPGMPALLSLVSLLSVLLMGCVAETG.

In some embodiments, the non-naturally occurring polypeptide of the present invention further comprises a hemagglutinin head domain sequence, or a variant thereof. In some embodiments, the hemagglutinin head domain sequence comprises the lateral patch sequence.

As used herein, the terms “influenza virus hemagglutinin head domain polypeptide,” “influenza virus hemagglutinin head domain,” “HA globular head domain,” and “HA head domain” refer to the globular head domain of an influenza hemagglutinin polypeptide. An influenza virus hemagglutinin head domain polypeptide or influenza virus hemagglutinin head domain may comprise or consist of a known (e.g., wild-type) influenza virus hemagglutinin head domain or may comprise or consist of a derivative, e.g. an engineered derivative, of a known (e.g., wild-type) influenza virus hemagglutinin head domain. Those of skill in the art will recognize that an influenza virus HA globular head domain typically comprises the amino acid residues intervening Cys that corresponds to amino acid position 52 of an influenza virus hemagglutinin HA1 domain of an H3 hemagglutinin (i.e., according to H3 numbering) and Cys that corresponds to amino acid position 277 of an influenza virus hemagglutinin HA1 domain of an H3 hemagglutinin (i.e., according to H3 numbering).

The invention also contemplates an influenza virus hemagglutinin head domain polypeptide derived from an influenza A virus of subtype H1, H2, H3, H4, H5, H6, H7, H8, H9, H10, H11, H12, H13, H14, H15, H16, H17, or H18.

The present invention relates to a stabilized eHead that target the lateral patch for H1 and H3.

Applicants used structure-guided protein design to extract the head region from the hemagglutinin protein (HA) and developed a stabilized eHead H1 immunogen. The eHead H1 construct includes HA residues from 55-263. Applicants employed glycan-masking to develop eHead variants focused on lateral patch epitope. In order to prime lateral patch responses consistently, Applicants also applied structure-guided mammalian-display directed evolution to redesign the eHead H1 to target human naive precursors of lateral patch bnAbs, in which the human precursors were identified in next-generation sequencing (NGS) data of human naive antibody heavy chains. To generate potent and long-lasting immune responses, Applicants also developed single-component self-assembling nanoparticles to provide multivalent display of the eHead. Applicants developed multiple types of nanoparticles: lumazine synthase-based 60mers, ferritin-based 24mers, non-ferritin based 24mers and 12mers. These nanoparticles are amenable to delivery by mRNA or by purified protein.

Applicants used structure-guided protein design to extract the head region from the hemagglutinin protein (HA) and developed a stabilized eHead H3 immunogen. The eHead H3 construct includes HA residues from 43-309. Applicants employed computational design approach and glycan-masking to develop a stabilized eHead H3 that is well-folded and highly expressed. In order to prime C585-like H3 lateral patch responses consistently, Applicants applied structure-guided mammalian-display directed evolution to redesign the eHead H3 to target C585-like human naive precursors. Compared to eHead H1, eHead H3 is more difficult to incorporate into nanoparticles. After a few rounds of iterative optimization, Applicants produced different 12mer, 24mer, and 60mer NPs for eHead. These NPs platforms are applied to the eHead H3 GT designs for priming C585-like responses.

Protein minimization and structural stabilization were employed in the design of eHead_H1 and eHead_H3.

A design of single-component self-assembling nanoparticles was employed for both the eHead H1 and H3.

In some embodiments, the polypeptide of the present invention comprises a glycosylase recognition sequence.

In some embodiments, the non-naturally occurring polypeptide of the present invention is glycosylated at one or more residues.

In some embodiments, the glycosylated polypeptide is glycosylated via N-linked glycosylation.

Glycan masking, in which N-linked glycosylation sites are added to regions outside the target epitope, in order to decrease off-target antibody response was utilized. Addition of glycans to proteins exposed on nanoparticles facilitates trafficking to germinal centers and thereby increase immune responses to target LP epitopes.

Germline-targeting vaccine design was employed to improve affinity for bnAb precursors. This increased the probability that priming immunization induces B cell responses with genetic and structural potential to mature into bnAbs.

The soluble eHead and nanoparticle constructs all use a secretion signal sequence from the pHLsec or pHLsecAvi vector (MGILPSPGMPALLSLVSLLSVLLMGCVAETG). Any secretion signal sequence suffices.

In some embodiments, the head domain sequence is an HINI head domain sequence. In some embodiments, the head domain sequence comprises amino acids 55-263 of hemagglutinin. In some embodiments, the head domain sequence comprises a variant comprising a deletion, a substitution, or an insertion of one or more amino acids, or a combination thereof.

In some embodiments, the variant head domain comprises a heterologous sequence comprising an RG sequence, an N sequence, an F sequence, a QDG sequence, or any combination thereof.

In some embodiments, the variant head domain comprises an RG substitution of a sequence comprising NK in the corresponding wild-type hemagglutinin polypeptide. In some embodiments, the variant head domain comprises at least 70%, 75%, 80%, 85%, 90%, 95%, or 99% identity to SEQ ID NO: 2. In some embodiments, the variant head domain comprises SEQ ID NO: 2.

In some embodiments, the variant head domain comprises an F substitution of a sequence comprising a Y in the corresponding wild-type hemagglutinin polypeptide. In some embodiments, the variant head domain comprises an N substitution of a sequence comprising an E in the corresponding wild-type hemagglutinin polypeptide. In some embodiments, the variant head domain comprises an N substitution of a sequence comprising an E in the corresponding wild-type hemagglutinin polypeptide and an F substitution of a sequence comprising a Y in the corresponding wilt-type hemagglutinin polypeptide. In some embodiments, the variant head domain comprises at least 70%, 75%, 80%, 85%, 90%, 95%, or 99% identity to SEQ ID NO: 3. In some embodiments, the variant head domain comprises SEQ ID NO: 3.

In some embodiments, the variant head domain comprises a QDG substitution of a sequence comprising an SRE sequence in the corresponding wild-type hemagglutinin polypeptide. In some embodiments, the variant head domain comprises an N substitution of a sequence comprising an E in the corresponding wild-type hemagglutinin polypeptide, an F substitution of a sequence comprising a Y in the corresponding wilt-type hemagglutinin polypeptide, and comprises a QDG substitution of a sequence comprising an SRE sequence in the corresponding wild-type hemagglutinin polypeptide. In some embodiments, the variant head domain comprises at least 70%, 75%, 80%, 85%, 90%, 95%, or 99% identity to SEQ ID NO: 4. In some embodiments, the variant head domain comprises SEQ ID NO: 4.

In some embodiments, the head domain sequence is an H3N2 head domain sequence. In some embodiments, the head domain sequence comprises amino acids 43-309 of hemagglutinin. In some embodiments, the head domain sequence comprises a variant comprising a deletion, a substitution, or an insertion of one or more amino acids or a combination thereof.

In some embodiments, the variant head domain comprises a heterologous sequence comprising an N sequence, an NST sequence, an NLT sequence, an NSS sequence, an E sequence, an SN sequence, an S sequence, an F sequence, a K sequence, a T sequence, a Y sequence, or any combination thereof.

In some embodiments, the variant head domain comprises an N substitution of a sequence comprising an S in the corresponding wild-type hemagglutinin polypeptide. In some embodiments, the variant head domain comprises a T substitution of a sequence comprising an S in the corresponding wild-type hemagglutinin polypeptide. In some embodiments, the variant head domain comprises an N substitution of a sequence comprising an R in the corresponding wild-type hemagglutinin polypeptide. In some embodiments, the variant head domain comprises an NST substitution of a sequence comprising an RSQ in the corresponding wild-type hemagglutinin polypeptide. In some embodiments, the variant head domain comprises an NLT substitution of a sequence comprising a VLV in the corresponding wild-type hemagglutinin polypeptide. In some embodiments, the variant head domain comprises an N substitution of a sequence comprising a K in the corresponding wild-type hemagglutinin polypeptide. In some embodiments, wherein the variant head domain comprises an NSS substitution of a sequence comprising an ISE in the corresponding wild-type hemagglutinin polypeptide. In some embodiments, the variant head domain comprises an E substitution of a sequence comprising an F in the corresponding wild-type hemagglutinin polypeptide. In some embodiments, the variant head domain comprises an SN substitution of a sequence comprising an NK in the corresponding wild-type hemagglutinin polypeptide. In some embodiments, the variant head domain comprises an S substitution of a sequence comprising a T in the corresponding wild-type hemagglutinin polypeptide. In some embodiments, the variant head domain comprises an N substitution of a sequence comprising an S in the corresponding wild-type hemagglutinin polypeptide, a T substitution of a sequence comprising an S in the corresponding wild-type hemagglutinin polypeptide, an N substitution of a sequence comprising an R in the corresponding wild-type hemagglutinin polypeptide, an NST substitution of a sequence comprising an RSQ in the corresponding wild-type hemagglutinin polypeptide, an NLT substitution of a sequence comprising a VLV in the corresponding wild-type hemagglutinin polypeptide, an N substitution of a sequence comprising a K in the corresponding wild-type hemagglutinin polypeptide, an NSS substitution of a sequence comprising an ISE in the corresponding wild-type hemagglutinin polypeptide, an E substitution of a sequence comprising an F in the corresponding wild-type hemagglutinin polypeptide, an SN substitution of a sequence comprising an NK in the corresponding wild-type hemagglutinin polypeptide, and an S substitution of a sequence comprising a T in the corresponding wild-type hemagglutinin polypeptide. In some embodiments, the variant head domain comprises a least 70%, 75%, 80%, 85%, 90%, 95%, or 99% identity to SEQ ID NO: 9. In some embodiments, the variant head domain comprises SEQ ID NO: 9.

In some embodiments, the variant head domain comprises an N substitution of a sequence comprising an S in the corresponding wild-type hemagglutinin polypeptide, an N substitution of a sequence comprising an R in the corresponding wild-type hemagglutinin polypeptide, an NST substitution of a sequence comprising an RSQ in the corresponding wild-type hemagglutinin polypeptide, an NLT substitution of a sequence comprising a VLV in the corresponding wild-type hemagglutinin polypeptide, and an N substitution of a sequence comprising a K in the corresponding wild-type hemagglutinin polypeptide. In some embodiments, the variant head domain comprises at least 70%, 75%, 80%, 85%, 90%, 95%, or 99% identity to SEQ ID NO: 10. In some embodiments, the variant head domain comprises SEQ ID NO: 10.

In some embodiments, the variant head domain comprises an F substitution of a sequence comprising a Y in the corresponding wild-type hemagglutinin polypeptide. In some embodiments, the variant head domain comprises a K substitution of a sequence comprising an E in the corresponding wild-type hemagglutinin polypeptide. In some embodiments, the variant head domain comprises a Y substitution of a sequence comprising a T in the corresponding wild-type hemagglutinin polypeptide. In some embodiments, the variant head domain comprises an F substitution of a sequence comprising a Y in the corresponding wild-type hemagglutinin polypeptide, a K substitution of a sequence comprising an E in the corresponding wild-type hemagglutinin polypeptide, and a Y substitution of a sequence comprising a T in the corresponding wild-type hemagglutinin polypeptide. In some embodiments, the variant head domain comprises at least 70%, 75%, 80%, 85%, 90%, 95%, or 99% identity to SEQ ID NO: 11. In some embodiments, the variant head domain comprises SEQ ID NO: 11.

In some embodiments the non-naturally occurring polypeptide of the present invention further comprises a tag. In some embodiments, the tag is an N-terminal tag. In some embodiments, the tag is a C-terminal tag. Examples of a tag include, but are not limited to, oligo-histidine (His), Avi-Tag (or avitag), glutathione-S-transferase, FLAG, streptavidin, streptag II, maltose binding protein, HA-tag, c-myc, T7-tag, S-tag, calmodulin binding peptide, fragment crystallizable (Fc) region of antibody, elastin like peptide, chitin-binding domain, thioredoxin, NusA, and albumin binding domain.

The eHead monomers also used a C-terminal purification tag (his-tag; GTKHHHHHH) or his-tag plus avitag (GTKHHHHHHGGSGGSGLNDIFEAQKIEWHE), but these are not required for protein folding and assembly.

In some embodiments, the non-naturally occurring polypeptide of the present invention further comprises one or more linkers. Any of the polypeptides disclosed herein may comprise a first linker sequence. Any of the polypeptides disclosed herein may comprise a second linker sequence. The first and second linker sequences may comprise the same sequence. The first and second linker sequences may comprise different sequences. The first and/or second linker sequences may be the same length. The first and/or second linker sequences may be different lengths. In some embodiments, the linker is linked to a C-terminus, a N-terminus, or both C-terminus and N-terminus.

The term “linker,” as used herein, refers to a chemical group or a molecule linking two molecules or moieties. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker may comprise a peptide or a non-peptide moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

In some embodiments, flexible linkers are contemplated. For example, GlySer linkers GGGS can be used. They can be used in repeats of 3 ((GGGGS)₃) or 6 ((GGGGS)₆), 9 ((GGGGS)₉) or even 12 ((GGGGS)₁₂) or more, to provide suitable lengths, as required. Other alternatives are (GGGGS)₁, (GGGGS)₂, (GGGGS)₄, (GGGGS)₅, (GGGGS)₇, (GGGGS)₈, (GGGGS)₁₀, or (GGGGS)₁₁. Additionally, XTEN linkers can be used, for example (XTEN), (XTEN-XTEN), or (XTEN-XTEN-XTEN). An alpha-helical linker such as (Ala(GluAlaAlaAlaLys)Ala) is also contemplated.

In some embodiments, rigid linkers are contemplated, such as (EAAAK)₃, (EAAAK)ₙ (n=1-3), A(EAAK)₄(ALEA(EAAAK)₄A, PAPAP, AEAAAKEAAAKA, (Ala-Pro)ₙ (10-34aa). In some embodiments, cleavable linkers are contemplated, such as, disulfide bonds, VSQTSKLTR|AETVFPDV, PLG|LWA, RVL|AEA, EDVVCC|SMSY, GGIER|GS, TRHRQPR|GWE, AGNRVRR|SVG, RRRRRRR|R|R.

In other embodiments, the linker is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides or at least 500 nucleotides in length.

One lumazine synthase platform uses a modified sequence derived from PDB ID: 1hqk (A. Aeolicus lumazine synthase).

Another lumazine synthase 60mer platform uses a modified sequence derived from PDB ID: 4v7g (Lumazine Synthase from Bacillus Anthracis)

The ferritin platform uses a modified sequence derived from PDB ID: 3bve (H. Pylori ferritin); Applicants also developed some glycan-masked 3bve nanoparticles.

The non-ferritin platform uses a modified sequence derived from PDB ID: 6ezj (Imidazoleglycerol-phosphate dehydratase).

The 12mer platform uses a modified sequence derived from PDB IDs: 2uyg, 2qqy, 4p6y, 2sd9, 6hsb, and 3n76.

Each of the nanoparticle platforms provide additional CD4 T help.

Additional broadly reactive CD4 T helper epitopes are contemplated.

Sequences for H1 are presented in Table 1.

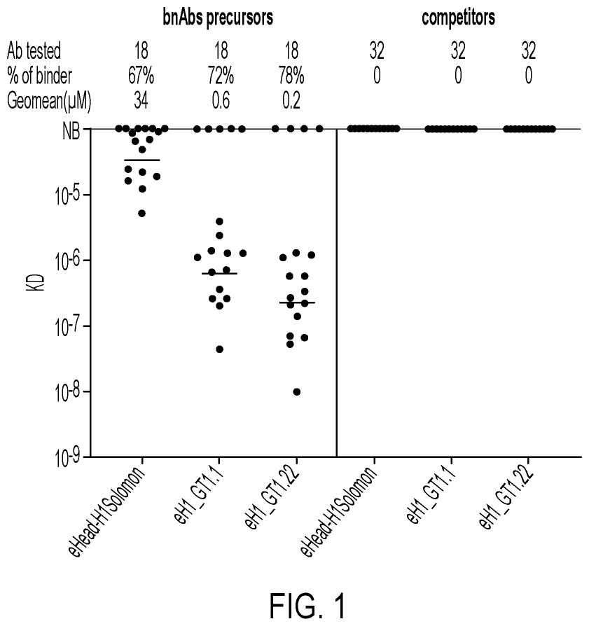

eHead-H1Solomon is the wt eHead sequence (residue 55-263 of H1 HA)A/Solomon Islands/3/2006]. eH1_GT1.1 is a germline-targeting immunogen that was developed by structure-guided mammalian display technique. It has the minimum of 2 mutation away from the wild type eHead sequence N174R, K175G underlined (RG). It has appreciable affinity to the lateral patch bnAb NGS precursors as shown in FIG. 1. GT1.22, with superior binding to lateral patch bnAb precursors, has one extra germline-targeting mutation E122N (underlined) and also has the Y98F mutation (underlined) to reduce sialic acid binding

eHead_H1_GT1_NP_v1 is a self-assembling 60mer nanoparticle in which the C-term of lumazine synthase (LS) (lowercase text) is fused to the N term of eH1_GT1.1 with GGS linker. Numbering starting at the lowercase text, an unpaired cysteine at position 37 was mutated to alanine (37A). A buried glycosylation site was mutated away at position 102 to aspartic acid (102D). Four cysteines were introduced (54C, 82C, 131C, 142C) to design two extra disulfide bridges. Three mutations were introduced (22A, 88S, 127A) to knock out the active sites of the nanoparticle. Amino acid substitutions into LS are indicated by uppercase letters. These constructs express well and have excellent nanoparticle assembly

eHead_H1_GTHaNP-v3 is a ferritin 24mer that uses GGS linker to fuse the N-term of ferritin (3bve) (lowercase) to the C-term of eH1_GT1.1. Numbering starting at the beginning of lowercase text, N18Q (uppercase) was introduced to remove the naturally occurring glycosylation site at position 18.

eHead_H1_3bve_g6 is a ferritin 24mer that add four extra glycans on the ferritin nanoparticle. Glycosylation sites beginning with N at positions 68, 78, 95, 104 are underlined. Substituted amino acids are in uppercase.

TABLE 1
H1 Sequences
monomer constructs

| | |
|---|---|
| SEQ 1: | MGILPSPGMPALLSLVSLLSVLLMGCVAETGAPLQLGNCSVAGWILGNPECELLISRESWS |
| eHead- | YIVEKPNPENGTCYPGHFADYEELREQLSSVSSFERFEIFPKESSWPNHTTTGVSASCSHNG |
| H1Solomon | ESSFYKNLLWLTGKNGLYPNLSKSYANNKEKEVLVLWGVHHPPNIGDQRALYHTENAYVSV |
| | VSSHYSRKFTPEIAKRPKVRDQEGRINYYWTLLEPGDTIIFEANGNLIAPRYAFALSRG |
| SEQ 2: | MGILPSPGMPALLSLVSLLSVLLMGCVAETGAPLQLGNCSVAGWILGNPECELLISRESWS |
| eH1_GT1.1 | YIVEKPNPENGTCYPGHFADYEELREQLSSVSSFERFEIFPKESSWPNHTTTGVSASCSHNG |
| | ESSFYKNLLWLTGKNGLYPNLSKSYANRGEKEVLVLWGVHHPPNIGDQRALYHTENAYVSV |
| | VSSHYSRKFTPEIAKRPKVRDQEGRINYYWTLLEPGDTIIFEANGNLIAPRYAFALSRG |
| SEQ 3: | MGILPSPGMPALLSLVSLLSVLLMGCVAETGAPLQLGNCSVAGWILGNPECELLISRESWS |
| eH1_GT1.22 | YIVEKPNPENGTCFPGHFADYEELREQLSSVSSFERFNIFPKESSWPNHTTTGVSASCSHN |
| | GESSFYKNLLWLTGKNGLYPNLSKSYANRGEKEVLVLWGVHHPPNIGDQRALYHTENAYV |
| | SVVSSHYSRKFTPEIAKRPKVRDQEGRINYYWTLLEPGDTIIFEANGNLIAPRYAFALSRG |

eH1_GT2.1_pHLsecAvi

| | |
|---|---|
| SEQ 4: | MGILPSPGMPALLSLVSLLSVLLMGCVAETGAPLQLGNCSVAGWILGNPECELLIQDGSWS |
| eH1_GT2.1 | YIVEKPNPENGTCFPGHFADYEELREQLSSVSSFERFNIFPKESSWPNHTTTGVSASCSHN |
| | GESSFYKNLLWLTGKNGLYPNLSKSYANRGEKEVLVLWGVHHPPNIGDQRALYHTENAYV |
| | SVVSSHYSRKFTPEIAKRPKVRDQEGRINYYWTLLEPGDTIIFEANGNLIAPRYAFALSRG |

Lumazine synthase nanoparticle for eH1_GT1.1

| | |
|---|---|
| SEQ 5: | MGILPSPGMPALLSLVSLLSVLLMGCVAETGmqiyegkltaeglrfgivasrAnhalvdrlvegaidAi |
| eHead_H1_GT1_NP_v1 | vrhggreeditlvrvCgsweipvaagelarkedidaviaigvlCrgatpSfdyiasevskglaDlslel |
| | rkpitfgvitadtleqaieAagtChgnkgweaalCaiemanlfkslrGGSGGSGGSGGSGGSGGSGGSG |
| | GGAPLQLGNCSVAGWILGNPECELLISRESWSYIVEKPNPENGTCFPGHFADYEELREQLSSVSSFE |
| | RFEIFPKESSWPNHTTTGVSASCSHNGESSFYKNLLWLTGKNGLYPNLSKSYANRGEKEVLV |
| | LWGVHHPPNIGDQRALYHTENAYVSVVSSHYSRKFTPEIAKRPKVRDQEGRINYYWTLLE |
| | PGDTIIFEANGNLIAPRYAFALSRG** |

Ferritin nanoparticles 24mer for eH1_GT1.1

| | |
|---|---|
| SEQ 6: | MGILPSPGMPALLSLVSLLSVLLMGCVAETGAPLQLGNCSVAGWILGNPECELLISRESWS |
| eHead_H1_GT1_NP_v3 | YIVEKPNPENGTCFPGHFADYEELREQLSSVSSFERFEIFPKESSWPNHTTTGVSASCSHN |
| | GESSFYKNLLWLTGKNGLYPNLSKSYANRGEKEVLVLWGVHHPPNIGDQRALYHTENAY |
| | VSVVSSHYSRKFTPEIAKRPKVRDQEGRINYYWTLLEPGDTIIFEANGNLIAPRYAFALSRG |
| | GGSGGSGGSGGSGGSlskdiikllneqvnkemQssnlymsmsswcythsldgaglflfdhaaeeyeha |
| | kkliiflnennvpvqltsisapehkfegltqifqkayeheqhisesinnivdhaikskdhatfnflqw |
| | yvaeqheeevlfkdildkielignenhglyladqyvkgiaksrks** |
| SEQ 7: | MGILPSPGMPALLSLVSLLSVLLMGCVAETGEPLQLGNCSVAGWILGNPECEHLISRESW |
| eHead_H1_3bve_g6_m | SSIIEKPNPENGTCFPGHFADYENLTENLSSVSSFERFEIFPKESSWPNHTTTGVSASCSHN |
| | GESSFYKNLLWLTGKNGLYPNLSKSYANNKEKEVLVLWGVHHPPNIGDQRALYHTENAY |
| | VSVNSSHYNRTFTPEIAKRPKVRDQEGRINYYWTLLEPGDTIIFEANGNLIAPRYAFALSRG |
| | GGSGGSGGSGGSGGSlskdiikllneqvnkemQssnlymsmsswcythsldgaglflfdhaaeeyeha |
| | kkliiflnennvpvNltsisapehNfTgltqifqkayeheqNisesinniTdhaikskdhatfnflqw |
| | yvaeqheeevlfkdildkielignenhglyladqyvkgiaksrks** |

Sequences for H3 are presented in Table 2. Signal sequences are italicized.

eHead-H3HK68 v1 is the wt eHead sequence for eHead H3 (residues 43-309 of H3 HA[A/Hong Kong/l/1968]). eH3_GT1.19 is a germline-targeting immunogen that was developed by structure-guided mammalian display. Compared to the wild type eHead H3, GT1.19 (comprising substitutions Y98F, E119K, T126Y, underlined) has improved binding to mature C585 bnAb (~100 fold increase) and the inferred germline version with mature HCDR3 (C585_CDR3mat_iGL, ~75 fold increase).

eHead_H3g93.1 is the stabilized glycan-masked eHead H3 design, including the following mutations (underlined in Table 2): S45N, S47T, S5193N, R201N, R208N, Q210T, V242N, V244T, K264N, I278N, E280S, F294E, N298S, K299N, and T301S. Comparing to the wild type eHead H3, it has improved the stability and expression yield and is able to form different nanoparticle platforms as shown in the sequence of eHead_H3_NP_v3.

eHead_H3g93.1_degly5 removes a few glycans from g3.1. Compared to eHead H3, it includes the following mutations (underlined in Table 2): S5193N, R201N, R208N, Q210T, V242N, V244T, and K264N.

TABLE 2
H3 Sequences
monomer constructs

| | |
|---|---|
| SEQ 8: | MGILPSPGMPALLSLVSLLSVLLMGCVAETGVQSSSTGKICNNPHRILDGIDCTLIDALLGD |
| eHead-H3HK68_v1 | PHCDVFQNETWDLFVERSKAFSNCYPYDVPDYASLRSLVASSGTLEFITEGFTWTGVTQN |
| | GGSNACKRGPGSGFFSRLNWLTKSGSTYPVLNVTMPNNDNFDKLYIWGVHHPSTNQEQ |
| | TSLYVQASGRVTVSTRRSQQTIIPNIGSRPWVRGLSSRISIYWTIVKPGDVLVINSNGNLIAP |
| | RGYFKMRTGKSSIMRSDAPIDTCISECITPNGSIPNDKPFQNVNKITYGACPKYV |
| SEQ 9: | MGILPSPGMPALLSLVSLLSVLLMGCVAETGVQNSTTGKICNNPHRILDGKNCTLIDALLG |
| eHead_H3_g3.1 | DPHCDNFSNETWDLFVERSKAYSNCYPYDVPDYASLRSLVASSGTLEFINESFTWTGVTQN |
| | GGSNACKRGPGSGFFSRLNWLTKNGTTYPVLNVTMPNNDSFDKLYIWGVHHPSTNQEQ |
| | TNLYVQASGNVTVSTRNSTQTIIPNIGSRPWVRGLSSRISIYWTIVKPGDNLTINSNGNLIAP |
| | RGYFKMRTGNSSIMRSDAPIDTCNSSCITPNGSIPNDKPEQNVSNISYGACPKYS |
| SEQ 10: | MGILPSPGMPALLSLVSLLSVLLMGCVAETGVQSSSTGKICNNPHRILDGKNCTLIDALLG |
| eHead_H3_g3.1_degly5 | DPHCDNFSNETWDLFVERSKAYSNCYPYDVPDYASLRSLVASSGTLEFINESFTWTGVTQN |
| | GGSNACKRGPGSGFFSRLNWLTKNGTTYPVLNVTMPNNDSFDKLYIWGVHHPSTNQEQ |
| | TNLYVQASGNVTVSTRNSTQTIIPNIGSRPWVRGLSSRISIYWTIVKPGDNLTINSNGNLIAP |
| | RGYFKMRTGNSSIMRSDAPIDTCISECITPNGSIPNDKPFQNVNKITYGACPKYS |
| SEQ 11: | MGILPSPGMPALLSLVSLLSVLLMGCVAETGVQSSSTGKICNNPHRILDGIDCTLIDALLGD |
| eH3_GT1.19 | PHCDVFQNETWDLFVERSKAFSNCFPYDVPDYASLRSLVASSGTLKFITEGFYWTGVTQN |
| | GGSNACKRGPGSGFFSRLNWLTKSGSTYPVLNVSMPNHDNFDKLYIWGVHHPSTNQEQ |
| | TSLYVQASGRVTVSTRRSQQTIIPNIGSRPWVRGLSSRISIYWTIVKPGDVLVINSNGNLIAP |
| | RGYFKMRTGKSSIMRSDAPIDTCISECITPNGSIPNDKPFQNVNKITYGACPKYV |

glycan masked resurfaced construct

| | |
|---|---|
| SEQ 12: | MGILPSPGMPALLSLVSLLSVLLMGCVAETGVQNSTSGSICLRPWRILDGKNCTLIDALLG |
| eH3_GT1.19_glyrsf_v2 | DPHCDNFSNSTWDLFVERSKAYSDCFPYDVPRYEDLRSLVAKSGTLKFITEGFYWTGVTQ |
| | NGTSNACKRGPNSSFFSRLNWLTKNGTSYPQLNVSMPNHDNFDKLYIWGVHHPSTNQE |
| | QTNLYVAESGNVTVSTSNSTQTIIPNIGSRPWVRGLSSRISIYWTIVKPGDVLVINSTGNLIA |
| | PRGYFKMREGNSSIMRSDAPTTNCNSSCITPNGSIDNSQPEQNVSNISYGDCPKPS |

LS 60mer of eHead_H3_g3.1

| | |
|---|---|
| SEQ 13: | MGILPSPGMPALLSLVSLLSVLLMGCVAETGMQIYEGKLTAEGLRFGIVASRANHALVDRL |
| eHead_H3_NP_v3 | VEGAIDAIVRHGGREEDITLVRVCGSWEIPVAAGELARKEDIDAVIAIGVLCRGATPSFDYI |
| | ASEVSKGLADLSLELRKPITFGVITADTLEQAIEAAGTCHGNKGWEAALCAIEMANLFKSL |
| | RGGSGGSGGSGGSGGSGGSGGSGGGVQNSTTGKICNNPHRILDGKNCTLIDALLGDPH |
| | CDNFSNETWDLFVERSKAYSNCFPYDVPDYASLRSLVASSGTLEFINESFTWTGVTQNGG |
| | SNACKRGPGSGFFSRLNWLTKNGTTYPVLNVTMPNNDSFDKLYIWGVHHPSTNQEQTN |
| | LYVQASGNVTVSTRNSTQTIIPNIGSRPWVRGLSSRISIYWTIVKPGDNLTINSNGNLIAPR |
| | GYFKMRTGNSSIMRSDAPIDTCNSSCITPNGSIPNDKPEQNVSNISYGACPKYS** |

ferritin 24mer of eHead_H3_g3.1

| | |
|---|---|
| SEQ 14: | MGILPSPGMPALLSLVSLLSVLLMGCVAETGVQNSTTGKICNNPHRILDGKNCTLIDALLG |
| eHead_H3_NP_v6 | DPHCDNFSNETWDLFVERSKAYSNCYPYDVPDYASLRSLVASSGTLEFINESFTWTGVTQN |
| | GGSNACKRGPGSGFFSRLNWLTKNGTTYPVLNVTMPNNDSFDKLYIWGVHHPSTNQEQ |
| | TNLYVQASGNVTVSTRNSTQTIIPNIGSRPWVRGLSSRISIYWTIVKPGDNLTINSNGNLIAP |
| | RGYFKMRTGNSSIMRSDAPIDTCNSSCITPNGSIPNDKPEQNVSNISYGACPKYSGGSGGS |
| | GGSGGSGGSLSKDIIKLLNEQVNKEMQSSNLYMSMSSWCYTHSLDGAGLFLFDHAAEEY |
| | EHAKKLIIFLNENNVPVQLTSISAPEHKFEGLTQIFQKAYEHEQHISESINNIVDHAIKSKDHA |
| | TFNFLQWYVAEQHEEEVLFKDILDKIELIGNENHGLYLADQYVKGIAKSRKS** |

12mer of eHead_H3_g3.1

| | |
|---|---|
| SEQ 15: | MGILPSPGMPALLSLVSLLSVLLMGCVAETGVQNSTTGKICNNPHRILDGKNCTLIDALLG |
| eHead_H3_NP_v7 | DPHCDNFSNETWDLFVERSKAYSNCYPYDVPDYASLRSLVASSGTLEFINESFTWTGVTQ |
| | NGGSNACKRGPGSGFFSRLNWLTKNGTTYPVLNVTMPNNDSFDKLYIWGVHHPSTNQ |
| | EQTNLYVQASGNVTVSTRNSTQTIIPNIGSRPWVRGLSSRISIYWTIVKPGDNLTINSNGN |
| | LIAPRGYFKMRTGNSSIMRSDAPIDTCNSSCITPNGSIPNDKPEQNVSNISYGACPKYSGG |
| | SGGSGGSGGSGGSNGSVLILNGPNLNLLGRREPEVYGNTTLEELNASAEAWGAELGLGV |
| | VFNQTNYEGQLIEWVQNASQEGFLAIVLNPGALTHYSYALLDAIRAQPLPVVEVHLTNLH |
| | AREEFRRHSVTAPAARGIVSGFGPLSYKLALVYLAETLEVGGEGF** |

different LS 60mer from 4v7g

| | |
|---|---|
| SEQ 16: | MGILPSPGMPALLSLVSLLSVLLMGCVAETGVQSSSTGKICNNPHRILDGKNCTLIDALLG |
| eHead_H3_NP_v22 | DPHCDNFSNETWDLFVERSKAYSNCYPYDVPDYASLRSLVASSGTLEFINESFTWTGVTQN |
| | GGSNACKRGPGSGFFSRLNWLTKNGTTYPVLNVTMPNNDSFDKLYIWGVHHPSTNQEQ |
| | TNLYVQASGNVTVSTRNSTQTIIPNIGSRPWVRGLSSRISIYWTIVKPGDNLTINSNGNLIAP |
| | RGYFKMRTGNSSIMRSDAPIDTCISECITPNGSIPNDKPFQNVNKITYGACPKYSGGSGGS |
| | GGSGGSGGSGGSGGSGGSVFEGHLVGTGLKVGVVVGRFNEFITSKLLGGALDGLKRHGV |
| | EENDIDVAWVPGAFEIPLIAKKMANSGKYDAVITLGTVIRGATTHYDYVANEVAKGVASLS |
| | LQTDIPVIFGVLTTETIEQAIERAGTKAGNKGYESAVAAIEMAHLSKHWA** |

non-ferritin 24mer platform

| | |
|---|---|
| SEQ 17: | MGILPSPGMPALLSLVSLLSVLLMGCVAETGVQSSSTGKICNNPHRILDGKNCTLIDALLG |
| eHead_H3_NP_v23 | DPHCDNFSNETWDLFVERSKAYSNCYPYDVPDYASLRSLVASSGTLEFINESFTWTGVTQN |
| | GGSNACKRGPGSGFFSRLNWLTKNGTTYPVLNVTMPNNDSFDKLYIWGVHHPSTNQEQ |
| | TNLYVQASGNVTVSTRNSTQTIIPNIGSRPWVRGLSSRISIYWTIVKPGDNLTINSNGNLIAP |
| | RGYFKMRTGNSSIMRSDAPIDTCISECITPNGSIPNDKPFQNVNKITYGACPKYSGGSGGS |
| | GGSGGSGGSGGSGGSGGSASPIESARIGEVKRETKETNVSVKINLDGHGVSDSSTGIPFLD |
| | HMLDQLASHGLFDVHVRATGDTHIDDHHTNEDVALAIGTALLKALGERKGINRFGDFTAP |
| | LDEALIHVSLDLSGRPYLGYNLEIPTQRVGTYDTQLVEHFFQSLVDTSGMTLHIRQLAGKNS |
| | HHIIEATFKAFARALRQATESDPRR** |

12mer derived from 2qqy

| | |
|---|---|
| SEQ 18: | MGILPSPGMPALLSLVSLLSVLLMGCVAETGVQSSSTGKICNNPHRILDGKNCTLIDALLG |
| eHead_H3_NP_v25 | DPHCDNFSNETWDLFVERSKAYSNCYPYDVPDYASLRSLVASSGTLEFINESFTWTGVTQN |
| | GGSNACKRGPGSGFFSRLNWLTKNGTTYPVLNVTMPNNDSFDKLYIWGVHHPSTNQEQ |
| | TNLYVQASGNVTVSTRNSTQTIIPNIGSRPWVRGLSSRISIYWTIVKPGDNLTINSNGNLIAP |
| | RGYFKMRTGNSSIMRSDAPIDTCISECITPNGSIPNDKPFQNVNKITYGACPKYSGGSGGS |
| | GGSGGSGGSSNAMSHDVKELIEGLNEDLAGEYSAIIMYNHNAATVSGIYRQVLKPFFESEI |
| | SDEQGHALYLAEKIKTLGGTPTTIPLRVKQAEDVREMLEYARQSEYETIKRYEKRKEQAANL |
| | NMTELVVKLEDMIADETNHMEELDRLLNDKAMVLN** |

12mer derived from 4p6y

| | |
|---|---|
| SEQ 19: | MGILPSPGMPALLSLVSLLSVLLMGCVAETGVQSSSTGKICNNPHRILDGKNCTLIDALLG |
| eHead_H3_NP_v26 | DPHCDNFSNETWDLFVERSKAYSNCYPYDVPDYASLRSLVASSGTLEFINESFTWTGVTQN |
| | GGSNACKRGPGSGFFSRLNWLTKNGTTYPVLNVTMPNNDSFDKLYIWGVHHPSTNQEQ |
| | TNLYVQASGNVTVSTRNSTQTIIPNIGSRPWVRGLSSRISIYWTIVKPGDNLTINSNGNLIAP |
| | RGYFKMRTGNSSIMRSDAPIDTCISECITPNGSIPNDKPFQNVNKITYGACPKYSGGSGGS |
| | GGSGGSGGSKELIRKLTEAFGPSGREEEVRSIILEELEGHIDGHRIDGLGNLIVWKGSGEKKV |
| | ILDAHIDEIGVVVTNVDDKGFLTIEPVGGVSPYMLLGKRIRFENGTIGVVGMEGETTEERQ |
| | ENVRKLSFDKLFIDIGANSREEAQKMAPIGSFGVYDSGFVEVSGKYVSKAMDDRIGAAVIV |
| | EVFKRIKPAVTLYGVFSVQEEVGLVGASVAGYGVPADEAIAIDVTDSADTPKAIKRHAMRLS |
| | GGPALKVKDRASISSKRILENLIEIAEKFDIKYQMEVLTFGGTNAMGYQRTREGIPSATVSIP |
| | TRYVHSPSEMIAPDDVEATVDLLIRYLGA** |

12mer derived from 2xd9

| | |
|---|---|
| SEQ 20: | MGILPSPGMPALLSLVSLLSVLLMGCVAETGVQSSSTGKICNNPHRILDGKNCTLIDALLG |
| eHead_H3_NP_v27 | DPHCDNFSNETWDLFVERSKAYSNCYPYDVPDYASLRSLVASSGTLEFINESFTWTGVTQN |
| | GGSNACKRGPGSGFFSRLNWLTKNGTTYPVLNVTMPNNDSFDKLYIWGVHHPSTNQEQ |
| | TNLYVQASGNVTVSTRNSTQTIIPNIGSRPWVRGLSSRISIYWTIVKPGDNLTINSNGNLIAP |
| | RGYFKMRTGNSSIMRSDAPIDTCISECITPNGSIPNDKPFQNVNKITYGACPKYSGGSGGS |
| | GGSGGSGGSMKILVIQGPNLNMLGHRDPRLYGMVTLDQIHEIMQTFVKQGNLDVELEFF |
| | QTNFEGEIIDKIQESVGSDYEGIIINPGAFSHTSIAIADAIMLAGKPVIEVHLTNIQAREEFRK |
| | NSYTGAAAGGVIMGFGPLGYNMALMAMVNILAEMKAFQEAQKNNPNNPINNQK** |

12mer derived from 6hsb

| | |
|---|---|
| SEQ 21: | MGILPSPGMPALLSLVSLLSVLLMGCVAETGVQSSSTGKICNNPHRILDGKNCTLIDALLG |
| eHead_H3_NP_v28 | DPHCDNFSNETWDLFVERSKAYSNCYPYDVPDYASLRSLVASSGTLEFINESFTWTGVTQN |
| | GGSNACKRGPGSGFFSRLNWLTKNGTTYPVLNVTMPNNDSFDKLYIWGVHHPSTNQEQ |
| | TNLYVQASGNVTVSTRNSTQTIIPNIGSRPWVRGLSSRISIYWTIVKPGDNLTINSNGNLIAP |
| | RGYFKMRTGNSSIMRSDAPIDTCISECITPNGSIPNDKPFQNVNKITYGACPKYSGGSGGS |
| | GGSGGSGGSAQIHVLVLHGPNLNLLGRREPDHYGRTTLAEIDARLKTEAEGRGWLLHSLQ |
| | SNAEHILVDAVQQAPTQGVTHIVLNPAAFTHTSVALRDALAAVAIPFIEVHLSNIHAREPFR |
| | RHSYFSDIASGLITGLGAEGYSLALDAIARRF** |

12mer derived from 3n76

| | |
|---|---|
| SEQ 22: | MGILPSPGMPALLSLVSLLSVLLMGCVAETGVQSSSTGKICNNPHRILDGKNCTLIDALLG |
| eHead_H3_NP_v29 | DPHCDNFSNETWDLFVERSKAYSNCYPYDVPDYASLRSLVASSGTLEFINESFTWTGVTQN |
| | GGSNACKRGPGSGFFSRLNWLTKNGTTYPVLNVTMPNNDSFDKLYIWGVHHPSTNQEQ |
| | TNLYVQASGNVTVSTRNSTQTIIPNIGSRPWVRGLSSRISIYWTIVKPGDNLTINSNGNLIAP |
| | RGYFKMRTGNSSIMRSDAPIDTCISECITPNGSIPNDKPFQNVNKITYGACPKYSGGSGGS |
| | GGSGGSGGSMSELIVNVINGPNLGRLGRREPAVYGGTTHDELVALIEREAAELGLKAVVRQ |
| | SDSEAQLLDWIHQAADAAEPVILNAGGLTHTSVALRDAAAELSAPLIEVHISNVHAREEFR |
| | RHSYLSPIATGVIVGLGIQGYLLALRYLAEHVGT** |

The present invention contemplates nanoparticles or nanoparticle formulations. The nanoparticle formulations may be a carbohydrate nanoparticle which may comprise a carbohydrate carrier and a modified nucleic acid molecule (e.g., mmRNA). As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; herein incorporated by reference in its entirety).

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

The average diameter of the nanoparticle employed in the compositions of the invention can be at least one member selected from the group consisting of about 20 nanometers, about 25 nanometers, about 30 nanometers, about 40 nanometers, about 50 nanometers, about 75 nanometers, about 100 nanometers, about 125 nanometers, about 150 nanometers, about 175 nanometers and about 200 nanometers. In another embodiment, the average diameter of the particle is at least one member selected from the group consisting of between about 10 to about 200 nanometers, between about 0.5 to about 5 microns and between about 5 to about 10 microns. In another embodiment, the average diameter of the microparticle is selected from the group consisting of about 0.1 μm, about 0.2 μm, about 0.4 μm, about 0.5 μm, about 1 μm and about 2 μm.

Nanoparticles for use in the compositions of the invention can be made from lipids or other fatty acids (see, for example, U.S. Pat. Nos. 5,709,879; 6,342,226; 6,090,406; Lian, et al., J. of Pharma. Sci. 90:667-680 (2001) and van Slooten, et al., Pharm Res. 17:42-48 (2000)) and non-lipid compositions (see, for example, Kreuter, J. Anat. 189:503-505 (1996), the teachings of all of which are hereby incorporated by reference in their entirety). The compositions can be bilayer or multilamellar liposomes and phospholipid based. Polymerized nanoparticles, as described, for example, in U.S. Pat. No. 7,285,289, the teachings of which are incorporated by reference in their entirety.

Metallic oxide nanoparticles for use in the compositions of the invention can be chemically substituted with at least one reactive moiety capable of forming a thioether bond employing conventionally techniques as described herein and in U.S. Pat. No. 6,086,881, the teachings of which are hereby incorporated by reference in their entirety. The antigen described herein can be coupled in a single step onto the metallic oxide particles by the formation of at least one thioether bond or it may be synthesized or assembled stepwise onto the metallic oxide particles after the initial thioether bond formation. The chemical derivatization reagents for the metallic oxide particles can include organosilane reagents that provide thioalkane functionality or other groups that may readily be converted into thiols or thiol-reactive moieties. Organosilane reagents which may be utilized for this purpose may be, but are not limited to, 3-mercaptopropyltrimethoxysilane, 3-aminopropyltriethoxysilane, 3-iodopropyltrimethoxysilane, 2-chloroethyltrichlorosilane, 3-glycidoxypropyltrimethoxysilane, vinyltrichlorosilane and 3-acryloxypropyltrimethoxysilane. Moieties that include one or more disulfide components may also be joined to the metallic oxide particle surface and thereby provide the corresponding reactive moiety able to enter into and form a thioether bond and juncture. Exemplary nanoparticles for use in the compositions of the invention include at least one member selected from the group consisting of poly (D,L-lactide-co-glycolide, also referred to as “poly(lactic-co-glycolic acid) and bisacyloxypropylcysteine.

Nanoparticles for use in the compositions of the invention can be made of inorganic material. Nanoparticles for use in the compositions of the invention can be made of a polymer material, such as at least one member selected from the group consisting of polystyrene, brominated polystyrene, polyacrylic acid, polyacrylonitrile, polyamide, polyacrylamide, polyacrolein, polybutadiene, polycaprolactone, polycarbonate, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze, a carbohydrate, carboxymethyl cellulose, hydroxyethyl cellulose, agar, gel, proteinaceous polymer, polypeptide, eukaryotic and prokaryotic cells, viruses, lipid, metal, resin, latex, rubber, silicone (e.g., polydimethyldiphenyl siloxane), glass, ceramic, charcoal, kaolinite and bentonite.

In some embodiments, a fusion polypeptide described herein further may comprise a self-assembling domain capable of forming a nanoparticle. In some embodiments, the self-assembling domain may comprise a type II 3-Dehydroquinase, ferritin or lumazine synthase. In some embodiments, the self-assembling domain may comprise a type II 3-Dehydroquinase polypeptide which may comprise one or more engineered glycosylation site. In some embodiments, the self-assembling domain may comprise a Thermus thermophilus type type II 3-Dehydroquinase, optionally which may comprise one or more engineered glycosylation site.

In some embodiments, the self-assembling domain may comprise a type II 3-Dehydroquinase, ferritin or lumazine synthase. In some embodiments, the self-assembling domain may comprise a Thermus thermophilus, Mycobacterium tuberculosis, Streptomyces coelicolor, Acinetobacter baumannii, Yersinia pestis, Bacillus subtilis, Proprionibacterium acnes, Acidithiobacillus caldus, Zymomonas mobilus, Helicobacter pylori, Pseudomonas aeruginosa, Candida albicans, or Psychromonas ingrahamii type II 3-Dehydroquinase polypeptide.

In some embodiments, the self-assembling domain may comprise a Thermus thermophilus type II 3-Dehydroquinase polypeptide. In some embodiments, the Thermus thermophilus type II 3-Dehydroquinase polypeptide may comprise an amino acid sequence having at least 70%, at least 80%, at least 85%, at least 90%, at least 95%, at least 97%, at least 98% at least 99% or at least 100% identity with MVLILNGPNLNLLGRREPEVYGRTTLEELEALCEAWGAELGLGVVFRQTNYEGQLIEW VQNAWQEGFLAIVLNPGALTHYSYALLDAIRAQPLPVVEVHLTNLHAREEFRRHSVTAP AARGIVSGFGPLSYKLALVYLAETLEVGGEGF. In some embodiments, the Thermus thermophilus type II 3-Dehydroquinase polypeptide may comprise the amino acid sequence of

MVLILNGPNLNLLGRREPEVYGRTTLEELEALCEAWGAELGLGVVFRQTN
YEGQLIEWVQNAWQEGFLAIVLNPGALTHYSYALLDAIRAQPLPVVEVHL
TNLHAREEFRRHSVTAPAARGIVSGFGPLSYKLALVYLAETLEVGGEGF.

In some embodiments, the 3-Dehydroquinase polypeptide may comprise one or more engineered glycosylation site, wherein the engineered glycosylation site may comprise the amino acid sequence of NXS or NXT, wherein X is not proline. In some embodiments, the one or more engineered glycosylation site is at an amino acid position corresponding to position 1, 25, 32, 49, and/or 63 of the 3-Dehydroquinase polypeptide may comprise the amino acid sequence of NGSVLILNGPNLNLLGRREPEVYGNTTLEELNASAEAWGAELGLGVVFNQTNYEGQLIE WVQNASQEGFLAIVLNPGALTHYSYALLDAIRAQPLPVVEVHLTNLHAREEFRRHSVTA PAARGIVSGFGPLSYKLALVYLAETLEVGGEGF. In some embodiments, the 3-Dehydroquinase polypeptide may comprise 1, 2, 3, 4, or 5 engineered glycosylation sites.

In some embodiments, the 3-Dehydroquinase polypeptide may comprise the amino acid sequence of

NGSVLILNGPNLNLLGRREPEVYGNTTLEELNASAEAWGAELGLGVVFNQ
TNYEGQLIEWVQNASQEGFLAIVLNPGALTHYSYALLDAIRAQPLPVVEV
HLTNLHAREEFRRHSVTAPAARGIVSGFGPLSYKLALVYLAETLEVGGEG
F.

In some embodiments, a fusion polypeptide described herein may comprise from the N terminus to the C terminus VP-SAD, SAD-VP, VP-SAD-VP, wherein VP and SAD corresponds to the at least one viral polypeptide, and self-assembling domain, respectively.

McKay et al. describe a self-amplifying RNA nanoparticle vaccine to immunize mice with saRNA encoding the SARS-CoV-2 spike protein encapsulated in LNP with doses ranging from 0.01 to 10 μg. (McKay et al., Self-amplifying RNA SARS-CoV-2 lipid nanoparticle vaccine induces equivalent preclinical antibody titers and viral neutralization to recovered COVID-19 patients. bioRxiv preprint doi: 10.1101/2020.04.22.055608 posted Apr. 25, 2020.) The methods disclosed can be used to express RBDs and proteins of the invention.

Moyer et al (NATURE MEDICINE|VOL 26|MARCH 2020|430-440) show how to add a phosphoserine motif to a subunit vaccine which then mediates binding to Alum, which this enhances in vivo trafficking to LNs and enhances immunogenicity.

Jardine et al (SCIENCE VOL 340 10 May 2013) report a design of a self-assembling nanoparticle presenting an engineered outer domain from HIV (eOD-GT6 60mer).

Sok et al. (SCIENCE 30 Sep. 2016 VOL 353 ISSUE 6307) show that the next generation version of this nanoparticle (eOD-GT8 60mer) induces responses from rare precursors in human-Ig-transgenic mice.

Kanekiyo et al. (NATURE IMMUNOLOGY|VOL 20|MARCH 2019|362-372) test purified protein subunit vaccines that are self-assembling nanoparticles (NPs) presenting RBDs from influenza hemagglutinin that elicit neutralizing responses.

Xu et al. describe a DNA vaccine comprising self-assembling nanoparticles comprising lumazine synthase for vaccination with an HIV immunogen and induction of strong humoral responses. (Xu et al., In Vivo Assembly of Nanoparticles Achieved through Synergy of Structure-Based Protein Engineering and Synthetic DNA Generates Enhanced Adaptive Immunity. Adv. Sci. 2020, DOI: 10.1002/advs.201902802.

Melo et al. describe an alphavirus RNA replicon for vaccination of subjects with germline-targeting HIV immunogens. (Melo et al., Immunogenicity of RNA Replicons Encoding HIV Env Immunogens Designed for Self-Assembly into Nanoparticles. Molecular Therapy, Vol. 27 No 12, pp. 1-11, December 2019).

In some embodiments, the viral polypeptide may comprise one or more engineered glycosylation sites.

N-linked glycosylation involves attachment of a carbohydrate consisting of several sugar molecules, sometimes also referred to as glycan, to the amide nitrogen of an asparagine (Asn) residue of a protein. This type of linkage is important for both the structure and function of many eukaryotic proteins. The N-linked glycosylation process occurs in eukaryotes and widely in archaea, but very rarely in bacteria. The nature of N-linked glycans attached to a glycoprotein is determined by the protein, and the cell in which it is expressed, and varies across species. The carbohydrate consists of sugar moieties, linked to one another in via glycosidic bonds. Attachment of a glycan residue to a protein requires the presence of the consensus sequence Asn-X-Ser/Thr wherein X is any amino acid except proline (Pro). Different species synthesize different types of N-linked glycan.

Further, in certain embodiments the antigen is engineered to display increased affinity. According to the invention, in certain embodiments, immunogens engineered for increased affinity are further engineered as to glycan masking. In a non-limiting embodiment, increased affinity immunogens, further comprise one, two, three, four, five, six or more additional glycosylation sites. In certain increased affinity immunogens, there may be found amino acid substitutions that remove native glycosylation sites. In such cases, glycosylation sites can optionally be restored so that on or more native glycans is maintained.

Generally, mammalian expression systems producing mammalian N-linked glycans are preferred, the goal being to promote neutralizing immune responses involving selected epitopes while minimize immunogenicity of epitopes that would elicit non-neutralizing antibodies. In certain instances, human expression systems such as HEK293 may be preferred, as animal cells such as CHO, Sp2/0 and NS0 mouse myeloma cells can produce glycoproteins with non-human glycans that may potentially illicit immunogenic responses.

Goh describes the types of host cells used for production of therapeutics, their glycosylation potential and the resultant impact on glycoprotein properties. Goh describes the various complex-type N-linked glycans and commonly used mammalian production cells, including Chinese hamster ovary (CHO) cells, baby hamster kidney (BHK) cells, NS0 myeloma and Sp2/0 hybridoma mouse cell lines, human embryonic kidney cells 293 (HEK293) and HT-1080 human cells. (Goh et al., Impact of host cell line choice on glycan profile. Critical Reviews in Biotechnology, 2008, 38:6, 851-867, DOI: 10.1080/07388551.2017.1416577). Lalonde reviews mammalian cell lines, including considerations of cell types, cell engineering, gene expression, cell growth and proliferation, protein folding and secretion. (Lalonde et al., Therapeutic glycoprotein production in mammalian cells. Journal of Biotechnology, 2017, 251:128; 10.1016/j.jbiotec.2017.04.028). The glycoprotein compostions are expressed similarly, taking in to account, for example, variations in types of glycosylation, protein expression and the like.

Croset examined differences in glycosylation in mammalian cells of 12 proteins in the two commonly used cell lines HEK and CHO. The cells were transiently transfected, and the expressed proteins were purified and analyzed on SDS-PAGE, isoelectric focusing (IEF), mass spectrometry and released glycans on capillary gel electrophoresis (CGE-LIF). For all proteins significant differences in the glycosylation were detected. The proteins migrated differently on SDS-PAGE, had different isoform patterns on IEF, showed different mass peak distributions on mass spectrometry and showed differences in the glycostructures detected in CGE. (Croset et al., Differences in the Glycosylation of Recombinant Proteins Expressed in HEK and CHO Cells. J Biotechnol. 2012 Oct. 31; 161(3):336-48. doi: 10.1016/j.jbiotec.2012.06.038. Epub 2012 Jul. 16.)

Clausen describes glycan structures and their metabolism, including state of glycosylation engineering in different cell types using precise gene-editing technologies (Clausen et al, Essentials of Glycobiology. 3rd edition. Varki A, Cummings R D, Esko J D, et al., editors. Cold Spring Harbor (NY): Cold Spring Harbor Laboratory Press; 2015-2017). Butler points out that although the glycosylation profile of these products is ‘human-like’ there is still the possibility of immunogenic epitopes such as α-Gal and Neu5Gc, and describes Human cell lines designed for high productivity of recombinant proteins and ensuring authentic glycosylation patterns. (Butler et al., The choice of mammalian cell host and possibilities for glycosylation engineering. Current Opinion in Biotechnology 2014, 30:107-112. DOI: 10.1016/j.copbio.2014.06.010).

Hunter reviews strategies for producing recombinant proteins in mammalian cell lines to introduce proper protein folding and post-translational modifications and how to overcome various obstacles that may be encountered. (Hunter et al., Optimization of Protein Expression in Mammalian Cells. Current Protocols, February 2019, Volume 95, Issue 1, p. e77. DOI:10.1002/cpps.77). Gupta reviews approaches that minimize the glycan heterogeneity for the production of the desired protein with improved glycoforms, including mammalian, insect, and yeast and glycoengineering to produce human-like glycan composition of a recombinant product. Gupta et al., Glycosylation control technologies for recombinant therapeutic proteins. Appl Microbiol Biotechnol 2018, 102, 10457-10468). The procedures and improvements described by Clausen and Hunter are used in expressing the glycoproteins of the invention

In some embodiments, the VP and SAD are directly linked, wherein VP and SAD corresponds to the at least one viral polypeptide, and self-assembling domain, respectively. In some embodiments, the fusion polypeptide may comprise one or more linkers linking the VP and SAD. In some embodiments, the fusion polypeptide may comprise one or more linkers linking the VP and SAD. In some embodiments, the one or more linker independently may comprise no more than 10 or no more than 5 amino acid residues. In some embodiments, the one or more linker independently may comprise one or more repeats of the GGS or GGGS sequence. In some embodiments, the one or more linker independently may comprise the amino acid sequence of

GGS, GGSGGS, GGSGGSGGS, GGGS, GGGSGGGS,
or
GGGSGGGSGGGS.

In some embodiments, a fusion polypeptide described herein further may comprise an amino acid sequence that targets the fusion polypeptide to the cell surface. In some embodiments, the amino acid sequence that targets the fusion polypeptide to the cell surface may comprise a GPI anchor signal sequence. In some embodiments, the amino acid sequence that targets the fusion polypeptide to the cell surface may comprise a transmembrane domain.

In some embodiments, the fusion polypeptide may comprise an amino acid sequence that targets the fusion polypeptide to the cell surface. In some embodiments, the amino acid sequence that targets the fusion polypeptide to the cell surface may comprise a GPI anchor signal sequence.

In some embodiments, the fusion polypeptide may comprise an amino acid sequence that targets the fusion polypeptide to the cell surface. In some embodiments, the amino acid sequence that targets the fusion polypeptide to the cell surface may comprise a transmembrane domain.

In some embodiments, the transmembrane domain (TM) is from HIV Env of the BG505 isolate (KIFIMIVGGLIGLRIVFAVLSVIHRVR), but any TM domain will suffice. For example, other TM domains could include the TM from SARS-CoV-2 (KWPWYIWLGFIAGLIAIVMVTIML) or the TM from VSV-G (KSSIASFFFIIGLIIGLFLVLR).

In some embodiments, the fusion polypeptide may comprise an amino acid sequence that targets the fusion polypeptide to the cell surface. transmembrane domain may comprise an HIV Env transmembrane domain, a SARS-CoV-2 transmembrane domain, or a VSV-G transmembrane domain. In some embodiments, the HIV Env transmembrane domain may comprise the amino acid sequence of KIFIMIVGGLIGLRIVFAVLSVIHRVR, the SARS-CoV-2 transmembrane domain may comprise the amino acid sequence of KWPWYIWLGFIAGLIAIVMVTIML, and the VSV-G transmembrane domain may comprise the amino acid sequence of KSSIASFFFIIGLIIGLFLVLR.

In some embodiments, the transmembrane domain may comprise a VSV-G transmembrane domain. In some embodiments, the VSV-G transmembrane domain may comprise the amino acid sequence of KSSIASFFFIIGLIIGLFLVLR.

In some embodiments, the viral polypeptide and the transmembrane domain are directly linked. In some embodiments, the viral polypeptide and the transmembrane domain are separated by a linker peptide. In some embodiments, the linker may comprise no more than 10 or no more than 5 amino acid residues. In some embodiments, the linker may comprise one or more repeats of the GGS or GGGS sequence. In some embodiments, the linker may comprise the amino acid sequence of GGS, GGSGGS, GGSGGSGGS, GGGS, GGGSGGGS, or GGGSGGGSGGGS.

In some embodiments, in a fusion polypeptide described herein the viral polypeptide is closer to the N terminus than the transmembrane domain.

In some embodiments, multimerization domains could be added in order to display clusters of the fusion polypeptide on the membrane surface. While tethering to the membrane already should provide a multivalent array of RBDs for B cell interaction, fusion to multimerization domains can enhance the local fusion polypeptide density and concomitantly enhance B cell activation. Such multimerization domains could be added either to a linker, or to the C-terminus of the construct after the TM domain. In some embodiments, the multimerization domain is added after the TM domain. Without being bound by any specific theory, this arrangement would hide the domain from B cell recognition and thus avoid generating non-RBD antibody responses. Examples of small multimerization domains (with fewer than 50 amino acids) include trimerization motifs like the coiled-coil GCN4 (PDB ID: 1GCN) or the trimeric fibritin foldon, or tetramerization motifs like the tetrameric variant of GCN4 in PDB ID 1GCL, or the heptameric coil in PDB ID: 4PNA or the octameric coil in PDB ID: 6G67. Larger multimerization domains with >100 amino acids which include a larger number of CD4 T helper epitopes could also be included. In some embodiments, a lumazine synthase domain that self-assembles into a pentamer can be fused C-terminal to the TM domain, to serve a dual purpose of providing additional T help and providing multimerization. Another example is the protein PH0250 that assembles into a 12-mer ring in PDB ID: 2EKD.

In an embodiment the invention provides an immunogenic or vaccine composition which may comprise a pharmaceutically or veterinarily acceptable carrier and an effective amount to elicit an immune response, or an effective amount to elicit a protective immune response, of: the non-naturally occurring immunogen, or the non-naturally nucleic acid molecule, or the vector, as disclosed herein.

In an embodiment, the vaccine or immunogenic composition may comprise or can be a subunit, DNA, DNA plasmid, mRNA, inactivated live chimeric (for example, but not limited to, chemical or ultraviolet inactivation), live chimeric; lyophilized; lyophilized and a constituent, readily water dissolvable, dispersing e.g. effervescent on admixing with water, in powder form, in tablet form, in liquid form, or aerosolized.

In an embodiment, the vaccine or immunogenic composition is or can be administered, without limitation, orally, nasally, perilingually, sublingually, rectally, subcutaneously, intradermally, or by injection.

In an embodiment, the vaccine or composition is or can be administered alone, as a single as a single administration; or administered as part of immunization/vaccination regimen such as bi-annually, annually, once or twice or thrice or quarterly or more such as monthly and/or a prime-boost regimen including where prime and boost same or different presentations of antigen (surface glycoprotein).

In an embodiment, the vaccine or composition is or can be administered in a regimen wherein regimen may comprise administration of one or more immunogenic or vaccine composition against another pathogen, e.g. influenza such as a coronavirus and influenza vaccination or immunization regimen.

In an embodiment, the vaccine or composition is or can be administered as part of a combination vaccine or co-administration or sequential administration with an immunogenic or vaccine composition against another pathogen, e.g. a combination of a coronavirus and influenza.

In an embodiment the invention provides a vaccine or immunogenic composition, including an adjuvant.

In an embodiment the invention provides in the composition including an adjuvant, the adjuvant may comprise aluminum hydroxide, or alum, or sodium bis(2-methoxyethoxy)aluminum hydride, or an oil-in-water adjuvant, water-in-oil adjuvant, or a carbomer adjuvant.

In an embodiment the invention provides a vaccine or immunogenic composition wherein the composition may comprise the non-naturally occurring immunogen, and the adjuvant may comprise aluminum hydroxide, or alum, or sodium bis(2-methoxyethoxy)aluminum hydride.

In an embodiment the invention provides a vaccine or immunogenic composition which may comprise the non-naturally occurring immunogen which may comprise the moiety capable of binding to a metal hydroxide adjuvant; or the moiety capable of binding to a metal hydroxide adjuvant at or near which may comprise within 25 amino acids of the N- or C-terminus; a moiety capable of binding to a metal hydroxide adjuvant which may comprise phosphoserine; or the moiety capable of binding to a metal hydroxide adjuvant at or near which may comprise within 25 amino acids of the N- or C-terminus which may comprise phosphoserine; or the moiety capable of binding to a metal hydroxide adjuvant which may comprise cysteine; or the moiety capable of binding to a metal hydroxide adjuvant at or near which may comprise within 25 amino acids of the N- or C-terminus which may comprise cysteine; wherein the adjuvant couples with the immunogen.

In some embodiments, nucleic acids encoding the non-naturally occurring polypeptide are envisioned. In some embodiments, the nucleic acid is DNA. In some embodiments, the nucleic acid is RNA. In some embodiments the nucleic acid is mRNA. In some embodiments, a vector comprising the nucleic acid encoding the non-naturally occurring polypeptide of the present invention is contemplated.

In some embodiments, a nanoparticle comprising the nucleic acid, polypeptide, and/or vector of the present invention is contemplated. In some embodiments, the nanoparticle is self-assembling.

In some embodiments, the nanoparticle comprises 24 subunits. In some embodiments, the nanoparticle comprises 12 subunits. In some embodiments, the nanoparticle comprises 70%, 75%, 80%, 85%, 90%, 95%, or 99% identity to SEQ ID NO: 15. In some embodiments, the nanoparticle comprises SEQ ID NO: 15.

In some embodiments, the nanoparticle comprises lumazine synthase. In some embodiments, the nanoparticle comprises 60 subunits of lumazine synthase. In some embodiments, the nanoparticle comprises 70%, 75%, 80%, 85%, 90%, 95%, or 99% identity to SEQ ID NO: 5. In some embodiments, the nanoparticle comprises SEQ ID NO: 5. In some embodiments, the nanoparticle comprises 70%, 75%, 80%, 85%, 90%, 95%, or 99% identity to SEQ ID NO: 13. In some embodiments, the nanoparticle comprises SEQ ID NO: 13.

In some embodiments, the nanoparticle comprises ferritin. In some embodiments, the nanoparticle comprises 24 subunits of ferritin. In some embodiments, the nanoparticle comprises 70%, 75%, 80%, 85%, 90%, 95%, or 99% identity to SEQ ID NO: 6. In some embodiments, the nanoparticle comprises SEQ ID NO: 6. In some embodiments, the nanoparticle comprises 70%, 75%, 80%, 85%, 90%, 95%, or 99% identity to SEQ ID NO: 7. In some embodiments, the nanoparticle comprises SEQ ID NO: 7. In some embodiments, the nanoparticle comprises 70%, 75%, 80%, 85%, 90%, 95%, or 99% identity to SEQ ID NO: 14. In some embodiments, the nanoparticle comprises SEQ ID NO: 14.

In some embodiments, a cell comprising the polypeptide, nucleic acid, vector and/or nanoparticle of the present invention is envisioned. In some embodiments, the cell is an immune cell. In some embodiments, the cell is a B cell.

In some embodiments, a pharmaceutical composition comprising the polypeptide, the nucleic acid, the vector, the nanoparticle, and/or the cell and a pharmaceutically acceptable excipient is envisioned. In some embodiments, a vaccine comprising the pharmaceutical composition of the present invention is envisioned. In some embodiments, a method of immunizing a subject in need thereof by administering the vaccine of the present invention is envisioned. In some embodiments, use of the pharmaceutical composition of the present invention to treat influenza infection is envisioned. In some embodiments, use of the vaccine of the present invention to prevent influenza infection is envisioned.

In an embodiment the invention provides a method for producing a non-naturally occurring immunogen as disclosed herein which may comprise expressing a non-naturally nucleic acid molecule as disclosed herein, or expressing a non-naturally nucleic acid molecule from a vector as disclosed herein; and optionally recovering, isolating and/or purifying the non-naturally occurring immunogen. Advantageously, for a subunit vaccine there is the recovering, isolating and/or purifying.

In an embodiment the invention provides a method for eliciting an immune or protective immune response in a mammal, or for eliciting, stimulating or producing an antibody or antibody response in a mammal, or for eliciting, stimulating or producing a neutralizing antibody (nAb) response in a mammal which may comprise administering an effective amount of the non-naturally occurring immunogen disclosed herein or a vaccine or immunogenic composition as disclosed herein; or, expressing in vivo a non-naturally occurring nucleic acid molecule herein disclosed, or, expressing in vivo a non-naturally nucleic acid molecule herein disclosed from a vector herein disclosed. In an embodiment the invention provides such a method wherein the mammal is a human, a non-human primate, a rodent, a chiroptera, or a bat, or a canine, or a dog, or a feline, or a cat, or a porcine, or a pig, or an equine, or a horse, or a bovine, or a cow or bull, or a mammal that may comprise elements of a human immune system. In an embodiment the invention provides such methods of the foregoing sentences of this paragraph wherein the mammal is capable of producing human antibodies.

Pre-clinical vaccine testing models, in particular the nucleic acid delivery systems, may be adapted to express the immunogens of the present invention.

J. Yu et al., Science 10.1126/science. abc6284 (2020) reports a naked DNA vaccine immunization in NHPs with various SARS-CoV-2 constructs, including a trimerized RBD, and tests protection against a virus challenge.

Corbett et al. (bioRxiv preprint https://doi.org/10.1101/2020.06.11.145920) report development of a Moderna mRNA vaccine called mRNA-1273 that encodes a stabilized SARS-CoV-2 spike which shows elicitation of nAbs and CD8 responses in mice.

McKay et al. describe a self-amplifying RNA nanoparticle vaccine to immunize mice with saRNA encoding the SARS-CoV-2 spike protein encapsulated in LNP with doses ranging from 0.01 to 10 μg. (McKay et al., Self-amplifying RNA SARS-CoV-2 lipid nanoparticle vaccine induces equivalent preclinical antibody titers and viral neutralization to recovered COVID-19 patients. bioRxiv preprint doi: 10.1101/2020.04.22.055608 posted Apr. 25, 2020.)

Erasmus describes a highly immunogenic vaccine candidate comprised of an RNA replicon (LION) designed to enhance vaccine stability, delivery, and immunogenicity delivery and immunogenicity for intramuscular injection to elicit antibody and T cell responses to SARS-CoV-2. (Erasmus et al., Single-dose replicating RNA vaccine induces neutralizing antibodies against SARS-CoV-2 in nonhuman primates. bioRxiv preprint doi: 10.1101/2020.05.28.121640 posted May 28, 2020). The methods disclosed can be used with the instant invention.

Smith et al (NATURE COMMUNICATIONS| (2020) 11:2601|https://doi.org/10.1038/s41467-020-16505-0|www.nature.com/naturecommunications) report mouse immunization results with the Inovio DNA vaccine encoding a SARS-CoV-2 spike protein.

Quinlan et al (bioRxiv preprint https://doi.org/10.1101/2020.04.10.036418doi) test a subunit vaccine composed of RBD-Fc conjugated to KLH carrier protein tests in rodents. Quinlan et al. show that the prototype elicits neutralizing antibodies and those antibodies do not mediate antibody-dependent enhancement (ADE) under conditions in which Zika virus ADE had previously been observed.

Ravichandran et al (bioRxiv preprint https://doi.org/10.1101/2020.05.12.091918) test three different subunit vaccines, including spike ectodomain (S1+S2), S1, and RBD, in rabbits and show that all three subunit vaccines elicit neutralizing Abs.

van Doremalen et al (bioRxiv preprint. https://doi.org/10.1101/2020.05.13.093195) report the Oxford vaccine that is being used by AstraZeneca (ChAdOx1 Chimpanzee Adenovirus vector (ChAd)) show that this vaccine provides some protection in NHPs.

Moyer et al (NATURE MEDICINE|VOL 26|MARCH 2020|430-440) show how to add a phosphoserine motif to a subunit vaccine which then mediates binding to Alum, which this enhances in vivo trafficking to LNs and enhances immunogenicity.

The invention also encompasses eliciting an immune response which may comprise systemically administering to an animal in need thereof an effective amount of any one of the non-naturally occurring protein(s) of the invention or a nucleic acid(s) encoding the same. The animal may be a mammal, advantageously a human.

In one embodiment, the nucleic acids of the present invention may be delivered as a therapeutic mRNA.

Provided herein are isolated nucleic acids (e.g., modified mRNAs encoding a peptide described herein) which may comprise a translatable region and at least two different nucleoside modifications, wherein the nucleic acid exhibits reduced degradation in a cell into which the nucleic acid is introduced, relative to a corresponding unmodified nucleic acid. For example, the degradation rate of the nucleic acid is reduced by at least 10%, 20%, 30%, 40%, 50%, 60%, 70%, 80%, or 90%, compared to the degradation rate of the corresponding unmodified nucleic acid. In certain embodiments, the nucleic acid may comprise RNA, DNA, TNA, GNA, or a hybrid thereof. In certain embodiments, the nucleic acid may comprise messenger RNA (mRNA). In certain embodiments, the mRNA does not substantially induce an innate immune response of the cell into which the mRNA is introduced. In certain embodiments, the mRNA may comprise at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In certain embodiments, the mRNA may comprise at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In other embodiments, the mRNA may comprise at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In yet other embodiments, the mRNA may comprise at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleic acids provided herein comprise a 5′ untranslated region (UTR) and/or a 3′UTR, wherein each of the two different nucleoside modifications are independently present in the 5′UTR and/or 3′UTR. In some embodiments, nucleic acids are provided herein, wherein at least one of the two different nucleoside modifications are present in the translatable region. In some embodiments, nucleic acids provided herein are capable of binding to at least one polypeptide that prevents or reduces an innate immune response of a cell into which the nucleic acid is introduced.

Further provided herein are isolated nucleic acids (e.g., modified mRNAs described herein) which may comprise (i) a translatable region encoding a peptide described herein, (ii) at least one nucleoside modification, and (iii) at least one intronic nucleotide sequence capable of being excised from the nucleic acid.

Further provided herein are isolated nucleic acids (e.g., modified mRNAs described herein) which may comprise (i) a translatable region encoding a peptide described herein, (ii) at least two different nucleoside modifications, and (iii) a degradation domain.

Further provided herein are non-enzymatically synthesized nucleic acids (e.g., modified mRNAs described herein) which may comprise at least one nucleoside modification, and which may comprise a translatable region encoding a peptide described herein. In certain embodiments, the non-enzymatically synthesized mRNA may comprise at least two different nucleoside modifications.

Further provided herein are isolated nucleic acids (e.g., modified mRNAs described herein) which may comprise a noncoding region and at least one nucleoside modification that reduces an innate immune response of a cell into which the nucleic acid is introduced, wherein the nucleic acid sequesters one or more translational machinery components. In certain embodiments, the isolated nucleic acids which may comprise a noncoding region and at least one nucleoside modification described herein are provided in an amount effective to reduce protein expression in the cell. In certain embodiments, the translational machinery component is a ribosomal protein or a transfer RNA (tRNA). In certain embodiments, the nucleic acid may comprise a small nucleolar RNA (sno-RNA), microRNA (miRNA), small interfering RNA (siRNA) or Piwi-interacting RNA (piRNA).

Further provided herein are isolated nucleic acids (e.g., modified mRNAs described herein) which may comprise (i) a first translatable region, (ii) at least one nucleoside modification, and (iii) an internal ribosome entry site (IRES). In certain embodiments, the IRES is obtained from a picornavirus, a pest virus, a polio virus, an encephalomyocarditis virus, a foot-and-mouth disease virus, a hepatitis C virus, a classical swine fever virus, a murine leukemia virus, a simian immune deficiency virus or a cricket paralysis virus. In certain embodiments, the isolated nucleic acid further may comprise a second translatable region. In certain embodiments, the isolated nucleic acid further may comprise a Kozak sequence. In some embodiments, the first translatable region encodes a peptide described herein. In some embodiments, the second translatable region encodes peptide described herein. In some embodiments, the first and the second translatable regions encode peptides described herein.

Provided herein are pharmaceutical compositions which may comprise: (i) an effective amount of a synthetic messenger ribonucleic acid (mRNA) encoding peptide described herein; and (ii) a pharmaceutically acceptable carrier, wherein i) the mRNA may comprise pseudouridine, 5′methyl-cytidine, or a combination thereof, or ii) the mRNA does not comprise a substantial amount of a nucleotide or nucleotides selected from the group consisting of uridine, cytidine, and a combination of uridine and cytidine, and wherein the composition is suitable for repeated administration (e.g., intravenous administration) to a mammalian subject in need thereof. In some embodiments,

Further provided herein are pharmaceutical compositions which may comprise and/or consisting essentially of: (i) an effective amount of a synthetic messenger ribonucleic acid (mRNA) encoding peptide described herein; (ii) a cell penetration agent; and (iii) a pharmaceutically acceptable carrier, wherein i) the mRNA may comprise pseudouridine, 5′methyl-cytidine or a combination thereof, or ii) the mRNA does not comprise a substantial amount of a nucleotide or nucleotides selected from the group consisting of uridine, cytidine, and a combination of uridine and cytidine, and wherein the composition is suitable for repeated administration (e.g., intravenous administration) to an animal (e.g., mammalian) subject in need thereof.

This invention provides nucleic acids, including RNAs such as mRNAs that contain one or more modified nucleosides (termed “modified nucleic acids”), which have useful properties including the lack of a substantial induction of the innate immune response of a cell into which the mRNA is introduced. Because these modified nucleic acids enhance the efficiency of protein production, intracellular retention of nucleic acids, and viability of contacted cells, as well as possess reduced immunogenicity, these nucleic acids having these properties are termed “enhanced nucleic acids” herein.

The term “nucleic acid,” in its broadest sense, includes any compound and/or substance that is or can be incorporated into an oligonucleotide chain. Exemplary nucleic acids for use in accordance with the present invention include, but are not limited to, one or more of DNA, RNA, hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc., described in detail herein.

Provided are modified nucleic acids containing a translatable region encoding a peptide described herein, and one, two, or more than two different nucleoside modifications. In some embodiments, the modified nucleic acid exhibits reduced degradation in a cell into which the nucleic acid is introduced, relative to a corresponding unmodified nucleic acid. For example, the degradation rate of the nucleic acid is reduced by at least 10%, 20%, 30%, 40%, 50%, 60%, 70%, 80%, or 90%, compared to the degradation rate of the corresponding unmodified nucleic acid. Exemplary nucleic acids include ribonucleic acids (RNAs), deoxyribonucleic acids (DNAs), threose nucleic acids (TNAs), glycol nucleic acids (GNAs), peptide nucleic acids (PNAs), locked nucleic acids (LNAs) or a hybrid thereof. In preferred embodiments, the modified nucleic acid includes messenger RNAs (mRNAs). As described herein, the nucleic acids of the invention do not substantially induce an innate immune response of a cell into which the mRNA is introduced.

In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In certain embodiments it is desirable to intracellularly degrade a modified nucleic acid introduced into the cell, for example if precise timing of protein production is desired. Thus, the invention provides a modified nucleic acid containing a degradation domain, which is capable of being acted on in a directed manner within a cell.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

Other components of nucleic acid are optional, and are beneficial in some embodiments. For example, a 5′ untranslated region (UTR) and/or a 3′UTR are provided, wherein either or both may independently contain one or more different nucleoside modifications. In such embodiments, nucleoside modifications may also be present in the translatable region. Also provided are nucleic acids containing a Kozak sequence.

Further, nucleic acids encoding a peptide described herein, and containing an internal ribosome entry site (IRES) are provided herein. An IRES may act as the sole ribosome binding site, or may serve as one of multiple ribosome binding sites of an mRNA. An mRNA containing more than one functional ribosome binding site may encode several peptides or polypeptides that are translated independently by the ribosomes (“multicistronic mRNA”). When nucleic acids are provided with an IRES, further optionally provided is a second translatable region. Examples of IRES sequences that can be used according to the invention include without limitation, those from picornaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

The therapeutic mRNAs as described, for example, in U.S. Pat. Nos. 9,464,124; 9,447,164; 9,428,535; 9,334,328; 9,303,079; 9,301,993; 9,295,689; 9,283,287; 9,271,996; 9,255,129; 9,254,311; 9,233,141; 9,221,891; 9,220,792; 9,220,755; 9,216,205; 9,192,651; 9,186,372; 9,181,319; 9,149,506; 9,114,113; 9,107,886; 9,095,552; 9,089,604; 9,061,059; 9,050,297; 8,999,380; 8,980,864; 8,822,663; 8,754,062; 8,710,200; 8,680,069 and 8,664,194 may be utilized for the present invention.

Methods for the chemical conjugation of polypeptides, carbohydrates, and/or lipids are well known in the art (see, for example, Hermanson. Bioconjugate Techniques (Academic Press; 1992); Aslam and Dent, eds. Bioconjugation: Protein coupling Techniques for the Biomedical Sciences (MacMillan: 1998); and Wong Chemistry of Protein Conjugation and Cross-linking (CRC Press: 1991)). For instance, primary amino groups may be incorporated by reaction with ethylenediamine in the presence of sodium cyanoborohydride and sulfhydryls may be introduced by reaction of cysteamin dihydrochloride followed by reduction with a standard disulfide reducing agent. Heterobifunctional crosslinkers, such as, for example, sulfosuccinimidyl (4-iodoacetyl) aminobenzoate, which link the epsilon amino group on the D-lysine residues of copolymers of D-lysine and D-glutamate to a sulfhydryl side chain from an amino terminal cysteine residue on the peptide to be coupled, may be used as well. Chemical conjugation also includes anything covalently bonded directly via side chain bonds or via a linker or spacer group.

It is noted that these therapeutics may be a chemical compound, a composition which may comprise a polypeptide of the present invention and/or antibody elicited by such a chemical compound and/or portion thereof or a pharmaceutically acceptable salt or a composition which may comprise a polypeptide of the invention, and may be administered alone or as an active ingredient in combination with pharmaceutically acceptable carriers, diluents, and vehicles, as well as other active ingredients.

The compounds or compositions may be administered orally, subcutaneously or parenterally including intravenous, intraarterial, intramuscular, intraperitoneally, and intranasal administration as well as intrathecal and infusion techniques.

It is noted that humans are treated generally longer than the mice or other experimental animals which treatment has a length proportional to the length of the disease process and drug effectiveness. The doses may be single doses or multiple doses over a period of several days, but single doses are preferred. Thus, one may scale up from animal experiments, e.g., rats, mice, and the like, to humans, by techniques from this disclosure and documents cited herein and the knowledge in the art, without undue experimentation.

The treatment generally has a length proportional to the length of the disease process and drug effectiveness and the patient being treated.

When administering a therapeutic of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier may be a solvent or dispersing medium containing, for example, water, ethanol, polyol (for example, glycerol, propylene glycol, liquid polyethylene glycol, and the like), suitable mixtures thereof, and vegetable oils.

Proper fluidity may be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions.

Additionally, various additives which enhance the stability, sterility, and isotonicity of the compositions, including antimicrobial preservatives, antioxidants, chelating agents, and buffers, may be added. Prevention of the action of microorganisms may be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form may be brought about by the use of agents delaying absorption, for example, aluminum monostearate and gelatin. According to the present invention, however, any vehicle, diluent, or additive used would have to be compatible with the compounds.

Sterile injectable solutions may be prepared by incorporating the compounds utilized in practicing the present invention in the required amount of the appropriate solvent with various amounts of the other ingredients, as desired.

A pharmacological formulation of the present invention, e.g., which may comprise a therapeutic compound or polypeptide of the present invention, may be administered to the patient in an injectable formulation containing any compatible carrier, such as various vehicles, adjuvants, additives, and diluents; or the compounds utilized in the present invention may be administered parenterally to the patient in the form of slow-release subcutaneous implants or targeted delivery systems such as monoclonal antibodies, iontophoretic, polymer matrices, liposomes, and microspheres.

A pharmacological formulation of the compound and composition which may comprise a polypeptide utilized in the present invention may be administered orally to the patient. Conventional methods such as administering the compounds in tablets, suspensions, solutions, emulsions, capsules, powders, syrups and the like are usable. Known techniques, which deliver the compound orally or intravenously and retain the biological activity, are preferred.

In one embodiment, a formulation of the present invention may be administered initially, and thereafter maintained by further administration. For instance, a formulation of the invention may be administered in one type of composition and thereafter further administered in a different or the same type of composition. For example, a formulation of the invention may be administered by intravenous injection to bring blood levels to a suitable level. The patient's levels are then maintained by an oral dosage form, although other forms of administration, dependent upon the patient's condition, may be used. In the instance of a vaccine composition, the vaccine may be administered as a single dose, or the vaccine may incorporate set booster doses. For example, booster doses may comprise variants in order to provide protection against multiple clades of SARS-CoV-2.

The quantity to be administered will vary for the patient being treated and whether the administration is for treatment or prevention and will vary from a few micrograms to a few milligrams for an average 70 kg patient, e.g., 5 micrograms to 5 milligrams such as 500 micrograms, or about 100 ng/kg of body weight to 100 mg/kg of body weight per administration and preferably will be from 10 pg/kg to 10 mg/kg per administration. Typically, however, the antigen is present in an amount on, the order of micrograms to milligrams, or, about 0.001 to about 20 wt %, preferably about 0.01 to about 10 wt %, and most preferably about 0.05 to about 5 wt %.

Of course, for any composition to be administered to an animal or human, including the components thereof, and for any particular method of administration, it is preferred to determine therefor: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable immunological response, such as by titrations of sera and analysis thereof for antibodies or antigens, e.g., by ELISA and/or RFFIT analysis. Such determinations do not require undue experimentation from the knowledge of the skilled artisan, this disclosure and the documents cited herein. And, the time for sequential administrations may be ascertained without undue experimentation. For instance, dosages may be readily ascertained by those skilled in the art from this disclosure and the knowledge in the art. Thus, the skilled artisan may readily determine the amount of compound and optional additives, vehicles, and/or carrier in compositions and to be administered in methods of the invention. Typically, an adjuvant or additive is commonly used as 0.001 to 50 wt % solution in phosphate buffered saline, and the active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %. Such determinations do not require undue experimentation from the knowledge of the skilled artisan, this disclosure and the documents cited herein. And, the time for sequential administrations may be ascertained without undue experimentation.

Examples of compositions which may comprise a therapeutic of the invention include liquid preparations for orifice, e.g., oral, nasal, anal, vaginal, peroral, intragastric, mucosal (e.g., perlingual, alveolar, gingival, olfactory or respiratory mucosa) etc., administration such as suspensions, syrups or elixirs; and, preparations for parenteral, subcutaneous, intradermal, intramuscular or intravenous administration (e.g., injectable administration), such as sterile suspensions or emulsions. Such compositions may be in admixture with a suitable carrier, diluent, or excipient such as sterile water, physiological saline, glucose or the like. The compositions may also be lyophilized. The compositions may contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Compositions of the invention, are conveniently provided as liquid preparations, e.g., isotonic aqueous solutions, suspensions, emulsions or viscous compositions which may be buffered to a selected pH. If digestive tract absorption is preferred, compositions of the invention may be in the “solid” form of pills, tablets, capsules, caplets and the like, including “solid” preparations which are time-released or which have a liquid filling, e.g., gelatin covered liquid, whereby the gelatin is dissolved in the stomach for delivery to the gut. If nasal or respiratory (mucosal) administration is desired, compositions may be in a form and dispensed by a squeeze spray dispenser, pump dispenser or aerosol dispenser. Aerosols are usually under pressure by means of a hydrocarbon. Pump dispensers may preferably dispense a metered dose or, a dose having a particular particle size.

Compositions of the invention may contain pharmaceutically acceptable flavors and/or colors for rendering them more appealing, especially if they are administered orally. The viscous compositions may be in the form of gels, lotions, ointments, creams and the like (e.g., for transdermal administration) and will typically contain a sufficient amount of a thickening agent so that the viscosity is from about 2500 to 6500 cps, although more viscous compositions, even up to 10,000 cps may be employed. Viscous compositions have a viscosity preferably of 2500 to 5000 cps, since above that range they become more difficult to administer. However, above that range, the compositions may approach solid or gelatin forms, which are then easily administered as a swallowed pill for oral ingestion.

Liquid preparations are normally easier to prepare than gels, other viscous compositions, and solid compositions. Additionally, liquid compositions are somewhat more convenient to administer, especially by injection or orally. Viscous compositions, on the other hand, may be formulated within the appropriate viscosity range to provide longer contact periods with mucosa, such as the lining of the stomach or nasal mucosa.

Obviously, the choice of suitable carriers and other additives will depend on the exact route of administration and the nature of the particular dosage form, e.g., liquid dosage form (e.g., whether the composition is to be formulated into a solution, a suspension, gel or another liquid form), or solid dosage form (e.g., whether the composition is to be formulated into a pill, tablet, capsule, caplet, time release form or liquid-filled form).

Solutions, suspensions and gels, normally contain a major amount of water (preferably purified water) in addition to the active compound. Minor amounts of other ingredients such as pH adjusters (e.g., a base such as NaOH), emulsifiers or dispersing agents, buffering agents, preservatives, wetting agents, jelling agents, (e.g., methylcellulose), colors and/or flavors may also be present. The compositions may be isotonic, i.e., it may have the same osmotic pressure as blood and lacrimal fluid.

The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions may be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Viscous compositions are normally prepared from solutions by the addition of such thickening agents.

A pharmaceutically acceptable preservative may be employed to increase the shelf-life of the compositions. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight although there may be appreciable variation depending upon the agent selected.

Those skilled in the art will recognize that the components of the compositions should be selected to be chemically inert with respect to the active compound. This will present no problem to those skilled in chemical and pharmaceutical principles, or problems may be readily avoided by reference to standard texts or by simple experiments (not involving undue experimentation), from this disclosure and the documents cited herein.

It is generally envisaged that compounds and compositions of the invention will be administered by injection, as such compounds are to elicit antibodies, and the skilled artisan may, from this disclosure and the knowledge in the art, formulate compounds and compositions identified by herein methods for administration by injection and administer such compounds and compositions by injection.

The inventive compositions of this invention are prepared by mixing the ingredients following generally accepted procedures. For example the selected components may be simply mixed in a blender, or other standard device to produce a concentrated mixture which may then be adjusted to the final concentration and viscosity by the addition of water or thickening agent and possibly a buffer to control pH or an additional solute to control tonicity. Generally the pH may be from about 3 to 7.5. Compositions may be administered in dosages and by techniques well known to those skilled in the medical arts taking into consideration such factors as the age, sex, weight, and condition of the particular patient, and the composition form used for administration (e.g., solid vs. liquid). Dosages for humans or other mammals may be determined without undue experimentation by the skilled artisan, from this disclosure, the documents cited herein, and the knowledge in the art.

Suitable regimes for initial administration and further doses or for sequential administrations also are variable, may include an initial administration followed by subsequent administrations; but nonetheless, may be ascertained by the skilled artisan, from this disclosure, the documents cited herein, and the knowledge in the art.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Development of Stabilized eHead that Target the Lateral Patch for H1

Applicants used structure-guided protein design to extract the head region from the hemagglutinin protein (HA) and developed a stabilized eHead H1 immunogen. The eHead H1 construct includes HA residues from 55-263. Applicants employed glycan-masking to develop eHead variants focused on lateral patch epitope. In order to prime lateral patch responses consistently, Applicants also applied structure-guided mammalian-display directed evolution to redesign the eHead H1 to target human naive precursors of lateral patch bnAbs, in which the human precursors were identified in next-generation sequencing (NGS) data of human naive antibody heavy chains. To generate potent and long-lasting immune responses, Applicants also developed single-component self-assembling nanoparticles to provide multivalent display of the eHead. Applicants developed multiple types of nanoparticles: lumazine synthase-based 60mers, ferritin-based 24mers, non-ferritin based 24mers and 12mers. These nanoparticles are amenable to delivery by mRNA or by purified protein.

### Example 2: Development of Stabilized eHead that Target the Lateral Patch for H3

Applicants used structure-guided protein design to extract the head region from the hemagglutinin protein (HA) and developed a stabilized eHead H3 immunogen. The eHead H3 construct includes HA residues from 43-309. Applicants employed computational design approach and glycan-masking to develop a stabilized eHead H3 that is well-folded and highly expressed. In order to prime C585-like H3 lateral patch responses consistently, Applicants applied structure-guided mammalian-display directed evolution to redesign the eHead H3 to target C585-like human naive precursors. Compared to eHead H1, eHead H3 is more difficult to incorporate into nanoparticles. After a few rounds of iterative optimization, Applicants were able to produce different 12mer, 24mer, and 60mer NPs for eHead. These NPs platforms will eventually be applied to the eHead H3 GT designs for priming C585-like responses.

### Example 3: Sequences for H1

TABLE 1 lists all of the following construct sequences.

### Monomer Constructs

eHead-H1Solomon is the wt eHead sequence (residue 55-263 of H1 HA)A/Solomon Islands/3/2006]. eH1_GT1.1 is a germline-targeting immunogen that was developed by structure-guided mammalian display technique. It has the minimum of 2 mutation away from the wild type eHead sequence N174R, K175G underlined (RG). It has appreciable affinity to the lateral patch bnAb NGS precursors as shown in FIG. 1. GT1.22, with superior binding to lateral patch bnAb precursors, has one extra germline-targeting mutation E122N (underlined) and also has the Y98F mutation (underlined) to reduce sialic acid binding (4).

eH1_GT2.1

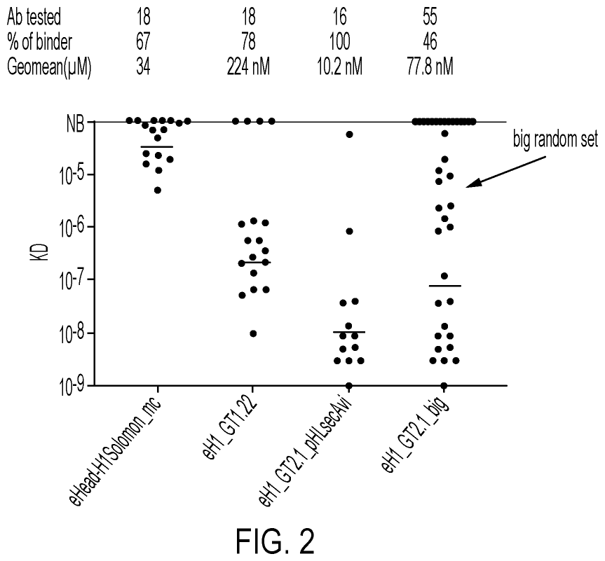

eH1_GT2.1 is a further improved germline-targeting immunogen which has better affinities to the LP NGS precursors (see FIG. 2). The new mutations are S79Q, R80D, and E81G underlined (QDG).

Lumazine Synthase Nanoparticle for eH1_GT1.1

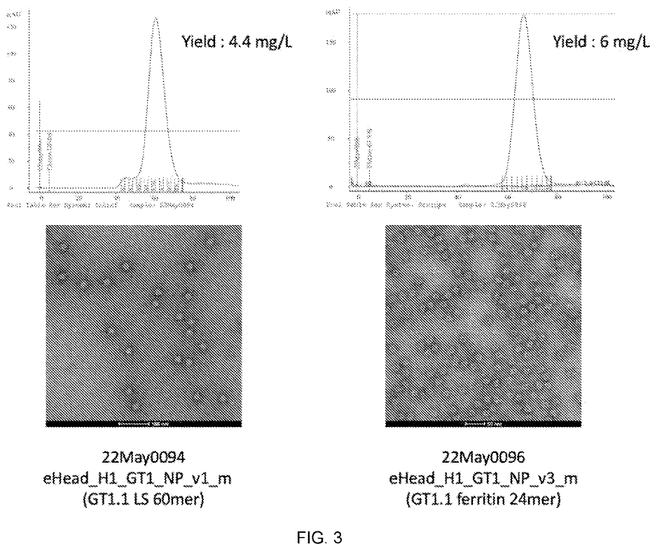

eHead_H1_GT1_NP_v1 is a self-assembling 60mer nanoparticle in which the C-term of lumazine synthase (LS) (lowercase text) is fused to the N term of eH1_GT1.1 with GGS linker. Numbering starting at the lowercase text, an unpaired cysteine at position 37 (underlined dark red) was mutated to alanine (37A). A buried glycosylation site was mutated away at position 102 to aspartic acid (102D). Four cysteines were introduced (54C, 82C, 131C, 142C) to design two extra disulfide bridges ((highlighted in yellow). Three mutations were introduced (22A, 88S, 127A) to knock out the active sites of the nanoparticle. Amino acid substitutions into LS are indicated by uppercase letters. These constructs express well and have excellent nanoparticle assembly (FIG. 3).

Ferritin Nanoparticles 24Mer for eH1_GT1.1

eHead_H1_GT1_NP_v3 is a ferritin 24mer that uses GGS linker to fuse the N-term of ferritin (3bve) (lowercase) to the C-term of eH1_GT1.1. Numbering starting at the beginning of lowercase text, N18Q (uppercase) was introduced to remove the naturally occurring glycosylation site at position 18.

eHead_H1_3bve_g6 is a ferritin 24mer that add four extra glycans on the ferritin nanoparticle. Glycosylation sites beginning with N at positions 68, 78, 95, 104 are underlined. Introduced amino acids are in uppercase.

### Example 6: Sequences for H3

TABLE 2 lists all of the following construct sequences.

### Monomer Constructs

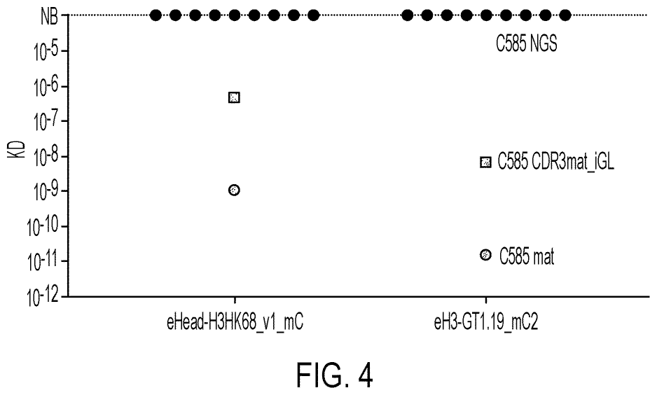

eHead-H3HK68_v1 is the wt eHead sequence for eHead H3 (residues 43-309 of H3 HA[A/Hong Kong/i/1968]). eH3_GT1.19 is a germline-targeting immunogen that was developed by structure-guided mammalian display. Compared to the wild type eHead H3, GT1.19 (comprising substitutions Y98F, E119K, T126Y) has improved binding to mature C585 bnAb (~100 fold increase) and the inferred germline version with mature HCDR3 (C585_CDR3mat_iGL, ~75 fold increase) (FIG. 4).

eHead_H3_g3.1 is the stabilized glycan-masked eHead H3 design, including the following mutations (underlined in Table 2): S45N, S47T, S193N, R201N, R208N, Q210T, V242N, V244T, K264N, I278N, E280S, F294E, N298S, K299N, and T301S. Comparing to the wild type eHead H3, it has improved the stability and expression yield and is able to form different nanoparticle platforms as shown in the sequence of eHead_H3_NP_v3.

eHead_H3_g3.1_degly5 removes a few glycans from g3.1. Compared to eHead H3, it includes the following mutations (underlined in Table 2): S193N, R201N, R208N, Q210T, V242N, V244T, and K264N.

### Glycan Masked Resurfaced Construct

The following nanoparticles use eHead_H3_g3.1_degly5 as the base construct to fuse to different nanoparticle platforms: eHead_H3_NP_v22, eHead_H3_NP_v23, eHead_H3_NP_v25, eHead_H3_NP_v26, eHead_H3_NP_v27, eHead_H3_NP_v28, eHead_H3_NP_v29. These platforms can be used for other immunogens.

The invention is further described by the following numbered paragraphs:
Paragraph 1. A non-naturally occurring polypeptide comprising an engineered hemagglutinin lateral patch sequence and a secretion signal sequence.
Paragraph 2. The polypeptide of paragraph 1, wherein the hemagglutinin lateral patch sequence, and the secretion signal sequence are operably linked.
Paragraph 3. The polypeptide of paragraph 1, wherein the secretion signal is N-terminal to the lateral patch sequence.
Paragraph 4. The polypeptide of paragraph 1, wherein the secretion signal is C-terminal to the lateral patch sequence.
Paragraph 5. The polypeptide of paragraph 1, wherein the polypeptide comprises a glycosylase recognition sequence.
Paragraph 6. The polypeptide of paragraph 1, wherein the secretion signal comprises at least 70%, 75%, 80%, 85%, 90%, 95%, or 99% identity to

MGILPSPGMPALLSLVSLLSVLLMGCVAETG.

Paragraph 7. The polypeptide of paragraph 1, wherein the secretion signal sequence comprises

MGILPSPGMPALLSLVSLLSVLLMGCVAETG.

Paragraph 8. The polypeptide of paragraph 1, further comprising a hemagglutinin head domain sequence, or a variant thereof.
Paragraph 9. The polypeptide of paragraph 8, wherein the hemagglutinin head domain sequence comprises the lateral patch sequence.
Paragraph 10. The polypeptide of paragraph 9, wherein the head domain sequence is an HINI head domain sequence.
Paragraph 11. The polypeptide of paragraph 10, wherein the head domain sequence comprises amino acids 55-263 of hemagglutinin.
Paragraph 12. The polypeptide of paragraph 11, wherein the head domain sequence comprises a variant comprising a deletion, a substitution, or an insertion, of one or more amino acids or a combination thereof.
Paragraph 13. The polypeptide of paragraph 12, wherein the variant head domain comprises a heterologous sequence comprising:
   (i) an RG sequence,
   (ii) an N sequence,
   (iii) an F sequence,
   (iv) a QDG sequence, or
   (v) any combination thereof.
Paragraph 14. The polypeptide of paragraph 13, wherein the variant head domain comprises an RG substitution of a sequence comprising NK in the corresponding wild-type hemagglutinin polypeptide.
Paragraph 15. The polypeptide of paragraph 14, wherein the variant head domain comprises at least 70%, 75%, 80%, 85%, 90%, 95%, or 99% identity to SEQ ID NO: 2
Paragraph 16. The polypeptide of paragraph 15, wherein the variant head domain comprises SEQ ID NO: 2
Paragraph 17. The polypeptide of paragraph 13, wherein the variant head domain comprises an F substitution of a sequence comprising a Y in the corresponding wild-type hemagglutinin polypeptide.
Paragraph 18. The polypeptide of paragraph 13, wherein the variant head domain comprises an N substitution of a sequence comprising an E in the corresponding wild-type hemagglutinin polypeptide.
Paragraph 19. The polypeptide of paragraph 13, wherein the variant head domain comprises an N substitution of a sequence comprising an E in the corresponding wild-type hemagglutinin polypeptide and an F substitution of a sequence comprising a Y in the corresponding wilt-type hemagglutinin polypeptide.
Paragraph 20. The polypeptide of paragraph 19, wherein the variant head domain comprises at least 70%, 75%, 80%, 85%, 90%, 95%, or 99% identity to SEQ ID NO: 3.
Paragraph 21. The polypeptide of paragraph 20, wherein the variant head domain comprises SEQ ID NO: 3.
Paragraph 22. The polypeptide of paragraph 13, wherein the variant head domain comprises a QDG substitution of a sequence comprising an SRE sequence in the corresponding wild-type hemagglutinin polypeptide.
Paragraph 23. The polypeptide of paragraph 13, wherein the variant head domain comprises an N substitution of a sequence comprising an E in the corresponding wild-type hemagglutinin polypeptide, an F substitution of a sequence comprising a Y in the corresponding wilt-type hemagglutinin polypeptide, and comprises a QDG substitution of a sequence comprising an SRE sequence in the corresponding wild-type hemagglutinin polypeptide.
Paragraph 24. The polypeptide of paragraph 23, wherein the variant head domain comprises at least 70%, 75%, 80%, 85%, 90%, 95%, or 99% identity to SEQ ID NO: 4.
Paragraph 25. The polypeptide of paragraph 24, wherein the variant head domain comprises SEQ ID NO: 4.
Paragraph 26. The polypeptide of paragraph 9, wherein the head domain sequence is an H3N2 head domain sequence.
Paragraph 27. The polypeptide of paragraph 26, wherein the head domain sequence comprises amino acids 43-309 of hemagglutinin.
Paragraph 28. The polypeptide of paragraph 27, wherein the head domain sequence comprises a variant comprising a deletion, a substitution, or an insertion, of one or more amino acids or a combination thereof.
Paragraph 29. The polypeptide of paragraph 28, wherein the variant head domain comprises a heterologous sequence comprising:
   (i) an N sequence,
   (ii) an NST sequence,
   (iii) an NLT sequence,
   (iv) an NSS sequence,
   (v) an E sequence,
   (vi) an SN sequence,
   (vii) an S sequence,
   (viii) an F sequence,
   (ix) a K sequence,
   (x) a T sequence,
   (xi) a Y sequence, or
   (xii) any combination thereof.
Paragraph 30. The polypeptide of paragraph 29, wherein the variant head domain comprises an N substitution of a sequence comprising an S in the corresponding wild-type hemagglutinin polypeptide.
Paragraph 31. The polypeptide of paragraph 29, wherein the variant head domain comprises a T substitution of a sequence comprising an S in the corresponding wild-type hemagglutinin polypeptide.
Paragraph 32. The polypeptide of paragraph 29, wherein the variant head domain comprises an N substitution of a sequence comprising an R in the corresponding wild-type hemagglutinin polypeptide.
Paragraph 33. The polypeptide of paragraph 29, wherein the variant head domain comprises an NST substitution of a sequence comprising an RSQ in the corresponding wild-type hemagglutinin polypeptide.
Paragraph 34. The polypeptide of paragraph 29, wherein the variant head domain comprises an NLT substitution of a sequence comprising a VLV in the corresponding wild-type hemagglutinin polypeptide.
Paragraph 35. The polypeptide of paragraph 29, wherein the variant head domain comprises an N substitution of a sequence comprising a K in the corresponding wild-type hemagglutinin polypeptide.
Paragraph 36. The polypeptide of paragraph 29, wherein the variant head domain comprises an NSS substitution of a sequence comprising an ISE in the corresponding wild-type hemagglutinin polypeptide.
Paragraph 37. The polypeptide of paragraph 29, wherein the variant head domain comprises an E substitution of a sequence comprising an F in the corresponding wild-type hemagglutinin polypeptide.
Paragraph 38. The polypeptide of paragraph 29, wherein the variant head domain comprises an SN substitution of a sequence comprising an NK in the corresponding wild-type hemagglutinin polypeptide.
Paragraph 39. The polypeptide of paragraph 29, wherein the variant head domain comprises an S substitution of a sequence comprising a T in the corresponding wild-type hemagglutinin polypeptide.
Paragraph 40. The polypeptide of paragraph 29, wherein the variant head domain comprises an N substitution of a sequence comprising an S in the corresponding wild-type hemagglutinin polypeptide, a T substitution of a sequence comprising an S in the corresponding wild-type hemagglutinin polypeptide, an N substitution of a sequence comprising an R in the corresponding wild-type hemagglutinin polypeptide, an NST substitution of a sequence comprising an RSQ in the corresponding wild-type hemagglutinin polypeptide, an NLT substitution of a sequence comprising a VLV in the corresponding wild-type hemagglutinin polypeptide, an N substitution of a sequence comprising a K in the corresponding wild-type hemagglutinin polypeptide, an NSS substitution of a sequence comprising an ISE in the corresponding wild-type hemagglutinin polypeptide, an E substitution of a sequence comprising an F in the corresponding wild-type hemagglutinin polypeptide, an SN substitution of a sequence comprising an NK in the corresponding wild-type hemagglutinin polypeptide, and an S substitution of a sequence comprising a T in the corresponding wild-type hemagglutinin polypeptide.
Paragraph 41. The polypeptide of paragraph 40, wherein the variant head domain comprises a least 70%, 75%, 80%, 85%, 90%, 95%, or 99% identity to SEQ ID NO: 9.
Paragraph 42. The polypeptide of paragraph 41, wherein the variant head domain comprises SEQ ID NO: 9.
Paragraph 43. The polypeptide of paragraph 29, wherein the variant head domain comprises an N substitution of a sequence comprising an S in the corresponding wild-type hemagglutinin polypeptide, an N substitution of a sequence comprising an R in the corresponding wild-type hemagglutinin polypeptide, an NST substitution of a sequence comprising an RSQ in the corresponding wild-type hemagglutinin polypeptide, an NLT substitution of a sequence comprising a VLV in the corresponding wild-type hemagglutinin polypeptide, and an N substitution of a sequence comprising a K in the corresponding wild-type hemagglutinin polypeptide.
Paragraph 44. The polypeptide of paragraph 43, wherein the variant head domain comprises at least 70%, 75%, 80%, 85%, 90%, 95%, or 99% identity to SEQ ID NO: 10.
Paragraph 45. The polypeptide of paragraph 44, wherein the variant head domain comprises SEQ ID NO: 10.
Paragraph 46. The polypeptide of paragraph 29, wherein the variant head domain comprises an F substitution of a sequence comprising a Y in the corresponding wild-type hemagglutinin polypeptide.
Paragraph 47. The polypeptide of paragraph 29, wherein the variant head domain comprises a K substitution of a sequence comprising an E in the corresponding wild-type hemagglutinin polypeptide.
Paragraph 48. The polypeptide of paragraph 29, wherein the variant head domain comprises a Y substitution of a sequence comprising a T in the corresponding wild-type hemagglutinin polypeptide.
Paragraph 49. The polypeptide of paragraph 29, wherein the variant head domain comprises an F substitution of a sequence comprising a Y in the corresponding wild-type hemagglutinin polypeptide, a K substitution of a sequence comprising an E in the corresponding wild-type hemagglutinin polypeptide, and a Y substitution of a sequence comprising a T in the corresponding wild-type hemagglutinin polypeptide.
Paragraph 50. The polypeptide of paragraph 49, wherein the variant head domain comprises at least 70%, 75%, 80%, 85%, 90%, 95%, or 99% identity to SEQ ID NO: 11 Paragraph 51. The polypeptide of paragraph 50, wherein the variant head domain comprises SEQ ID NO: 11.
Paragraph 52. The polypeptide of paragraph 1, wherein the polypeptide further comprises a tag.
Paragraph 53. The polypeptide of paragraph 52, wherein the tag is an N-terminal tag.
Paragraph 54. The polypeptide of paragraph 52, wherein the tag is a C-terminal tag.
Paragraph 55. The polypeptide of paragraph 1, wherein the polypeptide further comprises a linker.
Paragraph 56. The polypeptide of paragraph 1, wherein the polypeptide is glycosylated at one or more residues.
Paragraph 57. The polypeptide of paragraph 56, wherein the glycosylated polypeptide is glycosylated via N-linked glycosylation.
Paragraph 58. A nucleic acid encoding the polypeptide of any one of paragraphs 1-57.
Paragraph 59. The nucleic acid of paragraph 58, wherein the nucleic acid is DNA.
Paragraph 60. The nucleic acid of paragraph 58, wherein the nucleic acid is RNA.
Paragraph 61. The nucleic acid of paragraph 60, wherein the nucleic acid is mRNA.
Paragraph 62. A vector comprising the nucleic acid of paragraphs 58-61 Paragraph 63. A nanoparticle comprising the nucleic acid, polypeptide, and/or vector of any one of paragraphs 1-62.
Paragraph 64. The nanoparticle of paragraph 63, wherein the nanoparticle is a self-assembling nanoparticle.
Paragraph 65. The nanoparticle of paragraph 64, wherein the nanoparticle comprises 24 subunits.
Paragraph 66. The nanoparticle of paragraph 64, wherein the nanoparticle comprises 12 subunits.
Paragraph 67. The nanoparticle of paragraph 66, wherein the nanoparticle comprises 70%, 75%, 80%, 85%, 90%, 95%, or 99% identity to SEQ ID NO: 15.
Paragraph 68. The nanoparticle of paragraph 66, wherein the nanoparticle comprises SEQ ID NO: 15.
Paragraph 69. The nanoparticle of paragraph 64, wherein the nanoparticle comprises ferritin.
Paragraph 70. The nanoparticle of paragraph 69, wherein the nanoparticle comprises 24 subunits of ferritin.
Paragraph 71. The nanoparticle of paragraph 70, wherein the nanoparticle comprises 70%, 75%, 80%, 85%, 90%, 95%, or 99% identity to SEQ ID NO: 6.
Paragraph 72. The nanoparticle of paragraph 70, wherein the nanoparticle comprises SEQ ID NO: 6.
Paragraph 73. The nanoparticle of paragraph 70, wherein the nanoparticle comprises 70%, 75%, 80%, 85%, 90%, 95%, or 99% identity to SEQ ID NO: 7.
Paragraph 74. The nanoparticle of paragraph 70, wherein the nanoparticle comprises SEQ ID NO: 7.
Paragraph 75. The nanoparticle of paragraph 70, wherein the nanoparticle comprises 70%, 75%, 80%, 85%, 90%, 95%, or 99% identity to SEQ ID NO: 14.
Paragraph 76. The nanoparticle of paragraph 70, wherein the nanoparticle comprises SEQ ID NO: 14.
Paragraph 77. The nanoparticle of paragraph 64, wherein the nanoparticle comprises lumazine synthase.
Paragraph 78. The nanoparticle of paragraph 77, wherein the nanoparticle comprises 60 subunits of lumazine synthase.
Paragraph 79. The nanoparticle of paragraph 78, wherein the nanoparticle comprises 70%, 75%, 80%, 85%, 90%, 95%, or 99% identity to SEQ ID NO: 5.
Paragraph 80. The nanoparticle of paragraph 78, wherein the nanoparticle comprises SEQ ID NO: 5.
Paragraph 81. The nanoparticle of paragraph 78, wherein the nanoparticle comprises 70%, 75%, 80%, 85%, 90%, 95%, or 99% identity to SEQ ID NO: 13.
Paragraph 82. The nanoparticle of paragraph 78, wherein the nanoparticle comprises SEQ ID NO: 13.
Paragraph 83. A cell comprising the polypeptide, the nucleic acid, the vector, and/or the nanoparticle of any one of paragraphs 1-82.
Paragraph 84. The cell of paragraph 83, wherein the cell is an immune cell.
Paragraph 85. The cell of paragraph 84, wherein the immune cell is a B cell.
Paragraph 86. A pharmaceutical composition comprising the polypeptide, the nucleic acid, the vector, the nanoparticle and/or the cell of any one of paragraphs 1-85 and a pharmaceutically acceptable excipient.
Paragraph 87. A vaccine comprising the composition of paragraph 86.
Paragraph 88. A method for immunizing a subject in need thereof comprising administering an effective amount of the vaccine of paragraph 87.
Paragraph 89. Use of the pharmaceutical composition of paragraph 86 to treat influenza infection.
Paragraph 90. Use of the vaccine of paragraph 87 to prevent influenza infection.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A non-naturally occurring polypeptide comprising an engineered hemagglutinin lateral patch sequence and a secretion signal sequence.

2. The polypeptide of claim 1, wherein the hemagglutinin lateral patch sequence, and the secretion signal sequence are operably linked.

3. The polypeptide of claim 1, wherein the secretion signal is N-terminal to the lateral patch sequence.

4. The polypeptide of claim 1, wherein the secretion signal is C-terminal to the lateral patch sequence.

5. The polypeptide of claim 1, wherein the polypeptide comprises a glycosylase recognition sequence.

6. The polypeptide of claim 1, wherein the secretion signal comprises at least 70%, 75%, 80%, 85%, 90%, 95%, or 99% identity to MGILPSPGMPALLSLVSLLSVLLMGCVAETG.

7. The polypeptide of claim 1, wherein the secretion signal sequence comprises MGILPSPGMPALLSLVSLLSVLLMGCVAETG.

8. The polypeptide of claim 1, further comprising a hemagglutinin head domain sequence, or a variant thereof.

9. The polypeptide of claim 8, wherein the hemagglutinin head domain sequence comprises the lateral patch sequence.

10. The polypeptide of claim 9, wherein the head domain sequence is an H1N1 head domain sequence.

11. The polypeptide of claim 10, wherein the head domain sequence comprises amino acids 55-263 of hemagglutinin.

12. The polypeptide of claim 11, wherein the head domain sequence comprises a variant comprising a deletion, a substitution, or an insertion, of one or more amino acids or a combination thereof.

13. The polypeptide of claim 12, wherein the variant head domain comprises a heterologous sequence comprising:
(i) an RG sequence,
(ii) an N sequence,
(iii) an F sequence,
(iv) a QDG sequence, or
(v) any combination thereof

14. The polypeptide of claim 13, wherein the variant head domain comprises an RG substitution of a sequence comprising NK in the corresponding wild-type hemagglutinin polypeptide.

15. The polypeptide of claim 14, wherein the variant head domain comprises at least 70%, 75%, 80%, 85%, 90%, 95%, or 99% identity to SEQ ID NO: 2

16. The polypeptide of claim 15, wherein the variant head domain comprises SEQ ID NO: 2.

17. The polypeptide of claim 13, wherein the variant head domain comprises an F substitution of a sequence comprising a Y in the corresponding wild-type hemagglutinin polypeptide.

18. The polypeptide of claim 13, wherein the variant head domain comprises an N substitution of a sequence comprising an E in the corresponding wild-type hemagglutinin polypeptide.

19. The polypeptide of claim 13, wherein the variant head domain comprises an N substitution of a sequence comprising an E in the corresponding wild-type hemagglutinin polypeptide and an F substitution of a sequence comprising a Y in the corresponding wilt-type hemagglutinin polypeptide.

20. The polypeptide of claim 19, wherein the variant head domain comprises at least 70%, 75%, 80%, 85%, 90%, 95%, or 99% identity to SEQ ID NO: 3.

21. The polypeptide of claim 20, wherein the variant head domain comprises SEQ ID NO: 3.

22. The polypeptide of claim 13, wherein the variant head domain comprises a QDG substitution of a sequence comprising an SRE sequence in the corresponding wild-type hemagglutinin polypeptide.

23. The polypeptide of claim 13, wherein the variant head domain comprises an N substitution of a sequence comprising an E in the corresponding wild-type hemagglutinin polypeptide, an F substitution of a sequence comprising a Y in the corresponding wilt-type hemagglutinin polypeptide, and comprises a QDG substitution of a sequence comprising an SRE sequence in the corresponding wild-type hemagglutinin polypeptide.

24. The polypeptide of claim 23, wherein the variant head domain comprises at least 70%, 75%, 80%, 85%, 90%, 95%, or 99% identity to SEQ ID NO: 4.

25. The polypeptide of claim 24, wherein the variant head domain comprises SEQ ID NO: 4.

26. The polypeptide of claim 9, wherein the head domain sequence is an H3N2 head domain sequence.

27. The polypeptide of claim 26, wherein the head domain sequence comprises amino acids 43-309 of hemagglutinin.

28. The polypeptide of claim 27, wherein the head domain sequence comprises a variant comprising a deletion, a substitution, or an insertion, of one or more amino acids or a combination thereof.

29. The polypeptide of claim 28, wherein the variant head domain comprises a heterologous sequence comprising:
(i) an N sequence,
(ii) an NST sequence,
(iii) an NLT sequence,
(iv) an NSS sequence,
(v) an E sequence,
(vi) an SN sequence,
(vii) an S sequence,
(viii) an F sequence,
(ix) a K sequence,
(x) a T sequence,
(xi) a Y sequence, or
(xii) any combination thereof

30. The polypeptide of claim 29, wherein the variant head domain comprises an N substitution of a sequence comprising an S in the corresponding wild-type hemagglutinin polypeptide.

31. The polypeptide of claim 29, wherein the variant head domain comprises a T substitution of a sequence comprising an S in the corresponding wild-type hemagglutinin polypeptide.

32. The polypeptide of claim 29, wherein the variant head domain comprises an N substitution of a sequence comprising an R in the corresponding wild-type hemagglutinin polypeptide.

33. The polypeptide of claim 29, wherein the variant head domain comprises an NST substitution of a sequence comprising an RSQ in the corresponding wild-type hemagglutinin polypeptide.

34. The polypeptide of claim 29, wherein the variant head domain comprises an NLT substitution of a sequence comprising a VLV in the corresponding wild-type hemagglutinin polypeptide.

35. The polypeptide of claim 29, wherein the variant head domain comprises an N substitution of a sequence comprising a K in the corresponding wild-type hemagglutinin polypeptide.

36. The polypeptide of claim 29, wherein the variant head domain comprises an NSS substitution of a sequence comprising an ISE in the corresponding wild-type hemagglutinin polypeptide.

37. The polypeptide of claim 29, wherein the variant head domain comprises an E substitution of a sequence comprising an F in the corresponding wild-type hemagglutinin polypeptide.

38. The polypeptide of claim 29, wherein the variant head domain comprises an SN substitution of a sequence comprising an NK in the corresponding wild-type hemagglutinin polypeptide.

39. The polypeptide of claim 29, wherein the variant head domain comprises an S substitution of a sequence comprising a T in the corresponding wild-type hemagglutinin polypeptide.

40. The polypeptide of claim 29, wherein the variant head domain comprises an N substitution of a sequence comprising an S in the corresponding wild-type hemagglutinin polypeptide, a T substitution of a sequence comprising an S in the corresponding wild-type hemagglutinin polypeptide, an N substitution of a sequence comprising an R in the corresponding wild-type hemagglutinin polypeptide, an NST substitution of a sequence comprising an RSQ in the corresponding wild-type hemagglutinin polypeptide, an NLT substitution of a sequence comprising a VLV in the corresponding wild-type hemagglutinin polypeptide, an N substitution of a sequence comprising a K in the corresponding wild-type hemagglutinin polypeptide, an NSS substitution of a sequence comprising an ISE in the corresponding wild-type hemagglutinin polypeptide, an E substitution of a sequence comprising an F in the corresponding wild-type hemagglutinin polypeptide, an SN substitution of a sequence comprising an NK in the corresponding wild-type hemagglutinin polypeptide, and an S substitution of a sequence comprising a T in the corresponding wild-type hemagglutinin polypeptide.

41. The polypeptide of claim 40, wherein the variant head domain comprises a least 70%, 75%, 80%, 85%, 90%, 95%, or 99% identity to SEQ ID NO: 9.

42. The polypeptide of claim 41, wherein the variant head domain comprises SEQ ID NO: 9.

43. The polypeptide of claim 29, wherein the variant head domain comprises an N substitution of a sequence comprising an S in the corresponding wild-type hemagglutinin polypeptide, an N substitution of a sequence comprising an R in the corresponding wild-type hemagglutinin polypeptide, an NST substitution of a sequence comprising an RSQ in the corresponding wild-type hemagglutinin polypeptide, an NLT substitution of a sequence comprising a VLV in the corresponding wild-type hemagglutinin polypeptide, and an N substitution of a sequence comprising a K in the corresponding wild-type hemagglutinin polypeptide.

44. The polypeptide of claim 43, wherein the variant head domain comprises at least 70%, 75%, 80%, 85%, 90%, 95%, or 99% identity to SEQ ID NO: 10.

45. The polypeptide of claim 44, wherein the variant head domain comprises SEQ ID NO: 10.

46. The polypeptide of claim 29, wherein the variant head domain comprises an F substitution of a sequence comprising a Y in the corresponding wild-type hemagglutinin polypeptide.

47. The polypeptide of claim 29, wherein the variant head domain comprises a K substitution of a sequence comprising an E in the corresponding wild-type hemagglutinin polypeptide.

48. The polypeptide of claim 29, wherein the variant head domain comprises a Y substitution of a sequence comprising a T in the corresponding wild-type hemagglutinin polypeptide.

49. The polypeptide of claim 29, wherein the variant head domain comprises an F substitution of a sequence comprising a Y in the corresponding wild-type hemagglutinin polypeptide, a K substitution of a sequence comprising an E in the corresponding wild-type hemagglutinin polypeptide, and a Y substitution of a sequence comprising a T in the corresponding wild-type hemagglutinin polypeptide.

50. The polypeptide of claim 49, wherein the variant head domain comprises at least 70%, 75%, 80%, 85%, 90%, 95%, or 99% identity to SEQ ID NO: 11

51. The polypeptide of claim 50, wherein the variant head domain comprises SEQ ID NO: 11.

52. The polypeptide of claim 1, wherein the polypeptide further comprises a tag.

53. The polypeptide of claim 52, wherein the tag is an N-terminal tag.

54. The polypeptide of claim 52, wherein the tag is a C-terminal tag.

55. The polypeptide of claim 1, wherein the polypeptide further comprises a linker.

56. The polypeptide of claim 1, wherein the polypeptide is glycosylated at one or more residues.

57. The polypeptide of claim 56, wherein the glycosylated polypeptide is glycosylated via N-linked glycosylation.

58. A nucleic acid encoding the polypeptide of any one of claims 1-57.

59. The nucleic acid of claim 58, wherein the nucleic acid is DNA.

60. The nucleic acid of claim 58, wherein the nucleic acid is RNA.

61. The nucleic acid of claim 60, wherein the nucleic acid is mRNA.

62. A vector comprising the nucleic acid of claims 58-61

63. A nanoparticle comprising the nucleic acid, polypeptide, and/or vector of any one of claims 1-62.

64. The nanoparticle of claim 63, wherein the nanoparticle is a self-assembling nanoparticle.

65. The nanoparticle of claim 64, wherein the nanoparticle comprises 24 subunits.

66. The nanoparticle of claim 64, wherein the nanoparticle comprises 12 subunits.

67. The nanoparticle of claim 66, wherein the nanoparticle comprises 70%, 75%, 80%, 85%, 90%, 95%, or 99% identity to SEQ ID NO: 15.

68. The nanoparticle of claim 66, wherein the nanoparticle comprises SEQ ID NO: 15.

69. The nanoparticle of claim 64, wherein the nanoparticle comprises ferritin.

70. The nanoparticle of claim 69, wherein the nanoparticle comprises 24 subunits of ferritin.

71. The nanoparticle of claim 70, wherein the nanoparticle comprises 70%, 75%, 80%, 85%, 90%, 95%, or 99% identity to SEQ ID NO: 6.

72. The nanoparticle of claim 70, wherein the nanoparticle comprises SEQ ID NO: 6.

73. The nanoparticle of claim 70, wherein the nanoparticle comprises 70%, 75%, 80%, 85%, 90%, 95%, or 99% identity to SEQ ID NO: 7.

74. The nanoparticle of claim 70, wherein the nanoparticle comprises SEQ ID NO: 7.

75. The nanoparticle of claim 70, wherein the nanoparticle comprises 70%, 75%, 80%, 85%, 90%, 95%, or 99% identity to SEQ ID NO: 14.

76. The nanoparticle of claim 70, wherein the nanoparticle comprises SEQ ID NO: 14.

77. The nanoparticle of claim 64, wherein the nanoparticle comprises lumazine synthase.

78. The nanoparticle of claim 77, wherein the nanoparticle comprises 60 subunits of lumazine synthase.

79. The nanoparticle of claim 78, wherein the nanoparticle comprises 70%, 75%, 80%, 85%, 90%, 95%, or 99% identity to SEQ ID NO: 5.

80. The nanoparticle of claim 78, wherein the nanoparticle comprises SEQ ID NO: 5.

81. The nanoparticle of claim 78, wherein the nanoparticle comprises 70%, 75%, 80%, 85%, 90%, 95%, or 99% identity to SEQ ID NO: 13.

82. The nanoparticle of claim 78, wherein the nanoparticle comprises SEQ ID NO: 13.

83. A cell comprising the polypeptide, the nucleic acid, the vector, and/or the nanoparticle of any one of claims 1-82.

84. The cell of claim 83, wherein the cell is an immune cell.

85. The cell of claim 84, wherein the immune cell is a B cell.

86. A pharmaceutical composition comprising the polypeptide, the nucleic acid, the vector, the nanoparticle and/or the cell of any one of claims 1-85 and a pharmaceutically acceptable excipient.

87. A vaccine comprising the composition of claim 86.

88. A method for immunizing a subject in need thereof comprising administering an effective amount of the vaccine of claim 87.

89. Use of the pharmaceutical composition of claim 86 to treat influenza infection.

90. Use of the vaccine of claim 87 to prevent influenza infection.
